(19) United States
(12) Patent Application Publication
(10) Pub. No.: US 2026/0007555 A1
(43) Pub. Date: Jan. 8, 2026

(54) CONTRACTURE REDUCING EXTREMITY OFFLOADING SYSTEM

(71) Applicant: MARCUS MEDICAL DEVICES, LLC, Corona Del Mar, CA (US)

(72) Inventor: Michael J. Marcus, Corona Del Mar, CA (US)

(21) Appl. No.: 19/257,023

(22) Filed: Jul. 1, 2025

Related U.S. Application Data
(60) Provisional application No. 63/667,326, filed on Jul. 3, 2024.

(51) Int. Cl. A61G 7/057; A61G 7/075

(52) U.S. Cl. CPC A61G 7/057; A61G 7/0755

(57) ABSTRACT

A extremity offloading system including a body with an outer surface, an inner surface, and a thickness extending between the outer surface and the inner surface. The outer surface can be at least partially round. The inner surface can define an aperture. The aperture can be configured to accept insertion of a leg or an ankle of a patient. The extremity offloading system can also include a contraction adjustment support with a convex bottom surface and a concave top surface. The concave top surface can be configured to receive at least a portion of the body.

### INCORPORATION BY REFERENCE TO RELATED APPLICATION

This application claims the benefit of U.S. Provisional Application No. 63/667,326, filed Jul. 3, 2024, which is hereby incorporated by reference in its entirety. Any and all applications, if any, for which a foreign or domestic priority claim is identified in the Application Data Sheet of the present application are hereby incorporated by reference in their entireties under 37 CFR 1.57. This application incorporates by reference in its entirety for all purposes and makes a part of the present application U.S. patent application Ser. No. 17/455,342, filed Nov. 17, 2021, now U.S. Pat. No. 11,877,960.

### BACKGROUND

Various extremity offloading systems have been used in the healthcare industry to try and prevent a deep tissue pressure injury (DTPI), such as decubitus ulcers or bedsores, in patients who are on bed rest or generally immobile, but with limited success. The risk for a DTPI can develop anywhere on the body and, in particular, at any bony prominence of the body, such as the foot or ankle. In some cases, patients can have low or no sensitivity in areas in their body, such that the patient can be unaware DTPIs may be developing, which can lead to further complications. Initially, the area of the body develops a redness, which if left to progress can develop into a blister. This blister can subsequently become infected, which can then become an ulceration or DTPI. Not only can the development of a DTPI be painful, but they more importantly may be particularly complex and may also cause further complications, such as infections, osteomyelitis, sepsis, limb loss, or possibly even death. These further complications for patients can often lead to protracted and expensive extended hospital stays. Thus, preventing hospital acquired pressure injuries (HAPIs) is prudent for both health and financial reasons. These HAPIs may range in severity depending upon the depth of the tissue damage. HAPIs affect approximately 2.5 million individuals every year in the United States acute care facilities. They also vary in location, such as the heel, ankle, or foot, depending on location of the ulceration. The heel is a commonly affected region. The heel accounts for about 25 to 30% of these injuries. The heel is a common site for pressure ulcers among diabetic patients. About 15% of individuals with diabetes will eventually undergo amputation of a lower limb due to complications related to diabetes. After amputation, approximately 30 to 50% of those patients will require an amputation of the opposite limb within 3 to 5 years. The life expectancy for individuals with diabetes can vary significantly depending on various factors, like age, overall health, and management of the disease. Generally, it is reported that diabetes can reduce life expectancy by an average of 6 to 10 years, but this can be lower or higher based on individual circumstances. The mortality rate for patients with diabetic foot ulcers can be significant, with studies suggesting that about 10 to 20% may die from related complications.

Treatment protocols may range from the use of topical antibiotics creams, oral antibiotics, IV medications, enzymatic debridement ointments. Further treatment may involve surgical debridement, grafting and use of vacuum assisted devices to heal the affected tissues. With severe tissue damage extending to the bone, surgical intervention may be required for limb salvage. These interventions can include ulceration excision, bone resection and skin flap closures. Some complicated cases may require prolonged hospitalizations with possible referral to skilled nursing facilities.

### SUMMARY

The systems, methods and devices of this disclosure each have several innovative aspects, no single one of which is solely responsible for the desirable attributes disclosed herein.

In one aspect, an extremity offloading system can include a body with an outer surface, an inner surface, and a thickness extending between the outer surface and the inner surface. The outer surface can be at least partially round. The inner surface can define an aperture. The aperture can be configured to accept insertion of a leg or an ankle of a patient.

In some examples, the body can include a proximal end and a distal end, wherein the aperture extends through the proximal end and the distal end. The proximal end and the distal end can each be flat. The aperture can be cylindrical. A diameter of the aperture at the proximal end and a diameter of the aperture at the distal end can each be greater than a diameter of the aperture at the middle of the body.

In some aspects, the inner surface can include a plurality of flutes. A length of each of the plurality of flutes can extend along a length of the aperture. Each of the plurality of flutes can be equally spaced about a circumference of the aperture. The plurality of flutes can include 10 flutes. The inner surface can include a plurality of portions between each of the plurality of flutes. Each of the plurality of portions can include a flat surface. Each flat surface can be configured to be in contact with the leg or the ankle of the patient. Corners between each of the plurality of flutes and each of the plurality of portions can be rounded.

In some examples, the body includes an opening extending through a side of the body. The opening can extend through a thickness of the body between the outer surface and the inner surface. The opening can be angled relative to a longitudinal axis of the body. The longitudinal axis can extend from a proximal end to a distal end of the body. The opening can be defined by a first edge and a second edge of the body. The first edge and the second edge can be parallel. The first edge and the second edge can each be beveled.

In some aspects, the extremity offloading system can include one or more sensors configured to measure one or more of movement, pressure, temperature, humidity, or at least one patient parameter. The one or more sensors can include at least an accelerometer, a gyroscope, and a temperature sensor. The body can include foam.

In some aspects, the extremity offloading system can include a body comprising an outer surface, an inner surface, and a thickness extending between the outer surface and the inner surface. The outer surface can be at least partially round. The inner surface can define an aperture. The aperture can be configured to accept insertion of a leg or an ankle of a patient. The extremity offloading system can further include a contraction adjustment support with a convex bottom surface and a concave top surface. The concave top surface can be configured to receive at least a portion of the body.

In some aspects, the contraction adjustment support is removably attached to the body. The concave top surface can include an adhesive that is configured to attach the concave top surface to the outer surface of the body. The adhesive can include tape, glue, hook and loop fasteners, magnetic fasteners, or snap fasteners. The contraction adjustment support can be configured to be positioned between the body and a surface on which the support rests. The convex bottom surface can be positioned on the surface on which the support rests. The contraction adjustment support can be configured to elevate the body between 3 mm to 4 mm from the surface on which the support rests. The contraction adjustment support can include foam.

In yet another aspect, the extremity offloading system can include a body comprising an outer surface, an inner surface, and a thickness extending between the outer surface and the inner surface. The outer surface can be at least partially round. The inner surface can define an aperture. The aperture can be configured to accept insertion of a leg or an ankle of a patient. The extremity offloading system can further include a foot support attached to the body by at least one strap. The foot support can be configured to support a foot of the patient.

In some aspects, the at least one strap comprises a first end attached to the body and a second end attached to the foot support. Each of the first end is attached to the body and the second end can be attached to the foot support with an adhesive. The adhesive can include tape, glue, hook and loop fasteners, magnetic fasteners, or snap fasteners. The at least one strap can include two straps, a first strap on a first side of an opening of the body and a second strap on a second side of the opening of the body. A first end of the at least one strap can be attached to a first side of the body and a second end of the at least one strap is attached on a second side of the body. The foot support can include a channel extending through a width of the foot support. The channel can be configured to receive the at least one strap. The at least one strap can be an adjustment mechanism. The foot support can include a convex bottom surface and a concave top surface. A top surface of the foot support can include a plurality of grooves. A length of each of the plurality of grooves can extend along a length of the top surface of the foot support. The foot support can include foam.

### BRIEF DESCRIPTION OF THE DRAWINGS

The foregoing and other features of the present disclosure will become more fully apparent from the following description and appended claims, taken in conjunction with the accompanying drawings. Throughout the drawings, reference numbers may be re-used to indicate correspondence between referenced elements. Understanding that these drawings depict only several embodiments in accordance with the disclosure and are not to be considered limiting on scope. In addition, various features of different disclosed embodiments can be combined to form additional embodiments, which are part of this disclosure. Further, one or more features or structures can be removed or omitted.

FIG. 1 illustrates a proximal perspective view of an extremity offloading system in accordance with certain embodiments.

FIG. 2 illustrates an axial view of the extremity offloading system of FIG. 1 in accordance with certain embodiments.

FIG. 3 illustrates a cross-sectional proximal view along the width of the extremity offloading system of FIGS. 1-2 in accordance with certain embodiments.

FIG. 4 illustrates a medial view of the extremity offloading system of FIGS. 1-3 in accordance with certain embodiments.

FIG. 5 illustrates an anterior view of the extremity offloading system of FIGS. 1-4 in accordance with certain embodiments.

FIG. 6 illustrates a cross-sectional medial view along the length of the extremity offloading system at line A-A of FIG. 5 in accordance with certain embodiments.

FIG. 7 illustrates an exploded view of the extremity offloading system of FIGS. 1-6 in accordance with certain embodiments.

FIG. 8A illustrates the extremity offloading system positioned on a leg of a patient in an open position in accordance with certain embodiments.

FIG. 8B illustrates the extremity offloading system positioned on a leg of a patient in a closed position in accordance with certain embodiments.

FIG. 9 illustrates a perspective view of another embodiment of a extremity offloading system in accordance with certain embodiments.

FIG. 10 illustrates a top view of the extremity offloading system of FIG. 9 in accordance with certain embodiments.

FIG. 11 illustrates a partial perspective view of the extremity offloading system of FIGS. 9-10 in accordance with certain embodiments.

FIG. 12 illustrates a front view of the extremity offloading system of FIGS. 9-11 in accordance with certain embodiments.

FIG. 13 illustrates a cross-sectional view along a length of the extremity offloading system at line 13-13 of FIG. 12 in accordance with certain embodiments.

FIG. 14 illustrates a side view of the extremity offloading system of FIGS. 9-13 in accordance with certain embodiments.

FIG. 15 illustrates a perspective view of yet another embodiment of a extremity offloading system with a circuit in accordance with certain embodiments.

FIG. 16 illustrates a perspective view of yet another embodiment of a extremity offloading system in accordance with certain embodiments.

FIG. 17 illustrates another perspective view of the extremity offloading system of FIG. 16 in accordance with certain embodiments.

FIG. 18 illustrates a front view of the extremity offloading system of FIGS. 16-17 in accordance with certain embodiments.

FIG. 19 illustrates a rear view of the extremity offloading system of FIGS. 16-18 in accordance with certain embodiments.

FIG. 20 illustrates a right side view of the extremity offloading system of FIGS. 16-19 in accordance with certain embodiments.

FIG. 21 illustrates a left side view of the extremity offloading system of FIGS. 16-20 in accordance with certain embodiments.

FIG. 22 illustrates a top view of the extremity offloading system of FIGS. 16-21 in accordance with certain embodiments.

FIG. 23 illustrates a bottom view of the extremity offloading system of FIGS. 16-22 in accordance with certain embodiments.

FIG. 24 illustrates a sectional view through the line 24-24 in FIG. 18 in accordance with certain embodiments.

FIG. 25 illustrates a sectional view through the line 25-25 in FIG. 22 in accordance with certain embodiments.

FIG. 26 illustrates a perspective view of yet another embodiment of a extremity offloading system in accordance with certain embodiments.

FIG. 27 illustrates another perspective view of the extremity offloading system of FIG. 26 in accordance with certain embodiments.

FIG. 28 illustrates a front view of the extremity offloading system of FIGS. 26-27 in accordance with certain embodiments.

FIG. 29 illustrates a rear view of the extremity offloading system of FIGS. 26-28 in accordance with certain embodiments.

FIG. 30 illustrates a right side view of the extremity offloading system of FIGS. 26-29 in accordance with certain embodiments.

FIG. 31 illustrates a left side view of the extremity offloading system of FIGS. 26-30 in accordance with certain embodiments.

FIG. 32 illustrates a top view of the extremity offloading system of FIGS. 26-31 in accordance with certain embodiments.

FIG. 33 illustrates a bottom view of the extremity offloading system of FIGS. 26-32 in accordance with certain embodiments.

FIG. 34 illustrates a sectional view through the line 34-34 in FIG. 28 in accordance with certain embodiments.

FIG. 35 illustrates a sectional view through the line 35-35 in FIG. 32 in accordance with certain embodiments.

FIG. 36 illustrates a perspective view of the extremity offloading system with a support in accordance with certain embodiments.

FIGS. 37 illustrates an exploded view of the extremity offloading system with the support of FIG. 36 in accordance with certain embodiments.

FIG. 38 illustrates a front view of the extremity offloading system of FIGS. 36-37 in accordance with certain embodiments.

FIG. 39 illustrates a sectional view through line 39-39 in FIG. 38 in accordance with certain embodiments.

FIG. 40 illustrates a side view of the extremity offloading system of FIGS. 36-39 in accordance with certain embodiments.

FIG. 41 illustrates a sectional view through line 41-41 in FIG. 40 in accordance with certain embodiments.

FIG. 42 illustrates a perspective view of the extremity offloading system with two supports in accordance with certain embodiments.

FIG. 43 illustrates a front view of the extremity offloading system with two supports in accordance with certain embodiments.

FIG. 44 illustrates an extremity offloading system with a foot support positioned on a patient in a first position in accordance with certain embodiments.

FIG. 45 illustrates the extremity offloading system with the foot support positioned on the patient in a second position in accordance with certain embodiments.

FIG. 46 illustrates a front view of the foot support of FIGS. 44 and 45 in accordance with certain embodiments.

FIG. 47 illustrates a side view of the foot support of FIGS. 44-46 in accordance with certain embodiments.

FIG. 48 illustrates a top view of the foot support of FIGS. 44-47 in accordance with certain embodiments.

FIG. 49 illustrates a bottom view of the foot support of FIGS. 44-48 in accordance with certain embodiments.

### DETAILED DESCRIPTION

Offloading can be an effective and efficient method of preventing and treating DTPIs, whether in home environments or clinical settings. This unfortunate complication can affect a large variety of patients that are being treated for a myriad of reasons. Prevention of this type of injury not only improves patient outcomes, but also reducing excess medical costs. Once DTPIs develop, efficient methods of offloading can also be important for proper healing. In more severe cases, which can require extensive surgical interventions for tissue injuries to the heel, foot, or ankle, offloading can also be mandatory. All kinds of patients, but especially those with compromised blood flow, can be particularly at risk for DTPI. In the current healthcare climate, the most common patient population being affected includes diabetics, patients with peripheral arterial disease, stroke patients, and any patient who requires prolonged immobilization. Often these patients suffer from neuropathic problems that prevent them from feeling or perceiving the injury. Furthermore, complications due to COVID-19 can further contribute to the risk and development of DTPIs.

There are various solutions used to offload pressure on extremities to prevent DTPI or to allow patients to heal post recovery from surgical intervention and especially to keep pressure or weight off the back of a patient's heel. For example, a pillow or cushion can be positioned underneath a patient's leg or ankle when the patient's leg is substantially horizontal to a surface (e.g. when a patient is laying or sitting in bed). The pillow or cushion can elevate the patient's foot and keep pressure or weight off the back of the patient's heel. However, it can be difficult to achieve the desired position of the pillow and the patient's leg. Moreover, the desired position can be difficult to maintain. Patient compliance can also be difficult to achieve when a pillow or cushion is used. For example, since there is nothing to secure the pillow or cushion to the patient, the patient can shift or move out of the desired position, thereby reducing the effectiveness of the procedure and possibly even causing further injury. Additionally, use of a pillow can be inefficient offloading such that a DPTI can still be developed or inadequately treated. Further research indicates pillow use has limitations and that traditional heel offloading devices function better for offloading. Pillows can be used for patients who do not move their legs, but they often collapse or shift, leaving the heel in contact with the bed surface, which increases the risk of ischemia and pressure injuries. Pillows also require constant rearrangement to maintain proper offloading, which makes them less reliable than dedicated traditional heel-offloading devices.

The National Pressure Injury Advisory Panel (NPIAP) released the Prevention and Treatment of Ulcers/Injuries: Clinical Practice Guideline on Feb. 27, 2025 (hereinafter, “NPIAP Guideline”). The NPIAP Guideline recommends elevating the heels of individuals at risk for pressure injuries, ensuring they do not touch the support surface. The NPIAP Guideline also recommends using traditional heel offloading devices based on the patient's mobility and activity level. The NPIAP Guideline also includes indirect evidence and clinical expertise that indicates elevating the heels of individuals at risk of pressure injuries using standard pillows or cushions with sufficient height to ensure the heels are not in contact with the support surface can be effective. However, the use of pillows or cushions are less reliable than traditional heel offloading devices, which care better at preventing contact with pressure surfaces. For example, pillows or cushions can collapse or shift, which can risk heel contact with the support surface and potentially lead to injuries. The use of pillows can also require frequent adjustments, making them unreliable compared to specific traditional offloading devices.

Furthermore, in addition to the limitations of pillows providing adequate offloading, pillows also carry substantial infection risks due to contamination. Hospital pillows frequently harbor pathogenic bacteria, even after disinfection. Studies show that 38% of disinfected pillows tested positive for pathogens, including MRSA, Staph, E. Coli, VRE, and C. difficle. Some pillows harbored multiple pathogens, despite cleaning protocols using quaternary ammonium solutions. 15% of pillows contained greater than 2 pathogens after cleaning and 3% of pillows contained greater than 3 pathogens even after cleaning. Thus, the use of pillows can pose infection risks even after cleaning. Thus, traditional heel offloading devices, such as heel-offloading boots, are better suited for heel offloading than pillows in order to reduce pressure. The NPIAP Guideline also includes studies that show that heel-offloading boots significantly reduce the incidence and severity of heel pressure injuries compared to pillows. However, there is low certainty of evidence that using a traditional heel offloading device, such as a boot, is appropriate and would need to match the patient's mobility level.

Some systems seek to address these problems by using traditional heel offloading devices, such as hoops, casts, braces, boots or other devices that strap to the leg of a patient. However, these traditional heel offloading devices can be cumbersome or complicated to apply to a patient, in particular if the traditional heel offloading device has numerous straps and/or multiple holes for straps. The complicated nature of the traditional heel offloading devices can also lead to inconsistent application or improper positioning of the patient. These traditional heel offloading devices can also be cumbersome in terms of storage, transport or in use. These traditional heel offloading devices can also require frequent monitoring or adjustment, which can require attention from a medical professional. Furthermore, there is an increasing recognition of medical device related tissue issues. For example, the use of straps or other means to secure a traditional heel offloading device to a patient can, by themselves, cause injury to the patient. Medical device related tissue injuries can occur if the straps are incorrectly used or applied. For example, the straps can be in the wrong position or may be too loose or too tight. In particular, non-ambulatory patients can be at risk of developing skin trauma or injuries from the straps of a traditional heel offloading device.

Further, these traditional heel offloading devices may not allow a patient any type of movement or if a patient moves that can diminish the effectiveness of the traditional heel offloading device. For example, a heel suspension hoop can hold a patient's leg in the proper position but if the patient needs to be moved or if a patient moves, the patient's leg must be removed from the heel suspension hoop. In other examples, a leg cast or brace can be used to keep pressure off the back of a heel, but such traditional heel offloading devices can be cumbersome or complicated to apply and maintain. In some cases, the traditional heel offloading devices can be difficult to remove or attach (which may have to be done numerous times to allow for inspections or further procedures to the affected area). Patients may attempt to move or walk with the cast or brace on, which can lead to accidents or falls. Furthermore, if the patient moves with those traditional heel offloading devices the traditional heel offloading device position may change, which reduces the effectiveness and can even promote further injury. For example, the traditional heel offloading device can cause at least one leg of the patient to be positioned at an angle to elevate the heel, which can lead to issues in other parts of the patient's body (such as undesired pressure in another portion of the body). Furthermore, these types of traditional heel offloading devices can be expensive and difficult to store. Additionally, healthcare professionals often delay deploying these devices, such as in reaction to signs of ulcer emerging, as opposed to applying these traditional heel offloading devices proactively to prevent ulcers from emerging. A further issue with these traditional heel offloading devices is that the traditional heel offloading devices can increase humidity in the area that the traditional heel offloading device is applied, such as at the heel, which can increase the risk of skin issues, such as maceration. These traditional heel offloading devices can also enclose the limb, which can be undesirable in providing adequate airflow, in accessing the area for further treatment or observation, and can further contribute to the risk of skin issues.

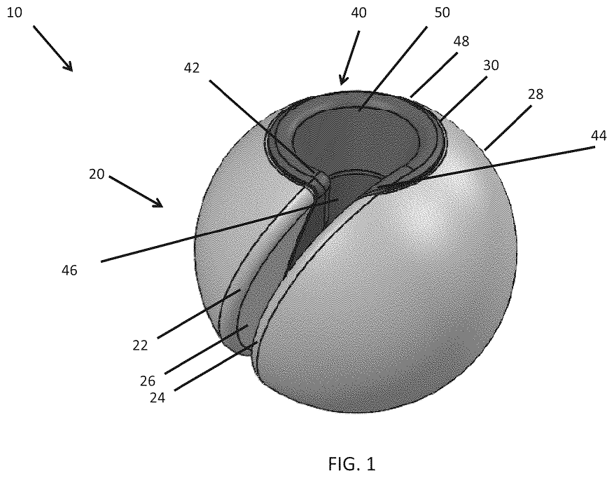

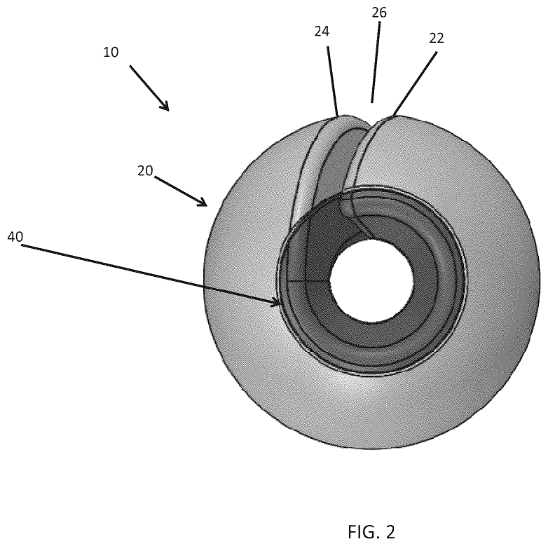

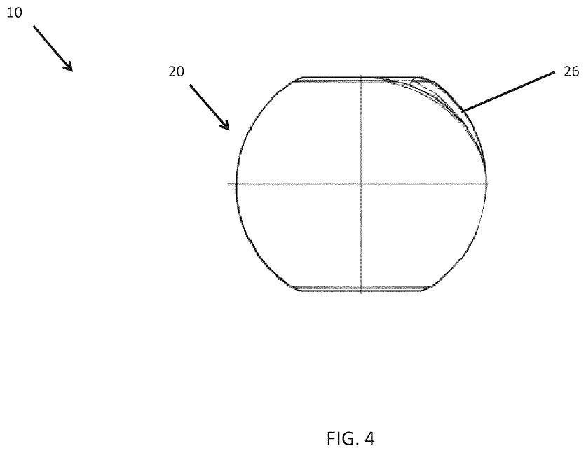

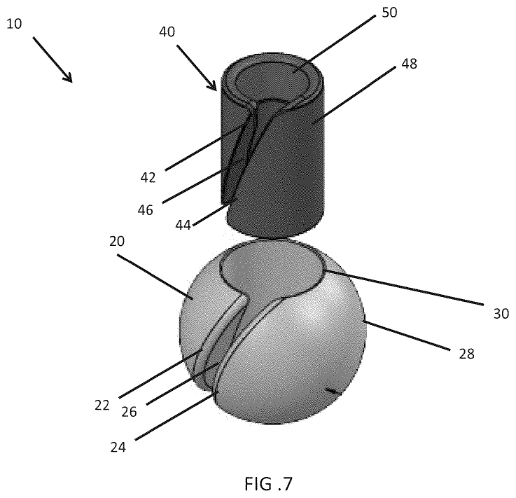

As shown in FIGS. 1-7, the extremity offloading system 10 includes a first component or portion 20 and a second component or portion 40. The extremity offloading system 10 can also be called a support system, a support assembly, a suspension system, a suspension assembly or a sphere for an extremity, limb, or heel. The first component 20 can be an outer component that is configured to receive or engage with the second component 40. Although described as being formed from two components 20 and 40, it should be understood that the extremity offloading system 10 can be formed from a single component (e.g., a unibody device) or formed from more than two components. The first component 20 can have an outer surface 28 that is spherical or round or at least partially spherical or round. As shown, the proximal end and the distal end can each be flat, such that the first component 20 does not form a full sphere. The proximal end can be considered the top end. The distal end can be considered the bottom end. Furthermore, in some embodiments, the proximal end and distal end may be reversed. The first component 20 can have a first thickness between the inner surface 30 and the outer surface 28.

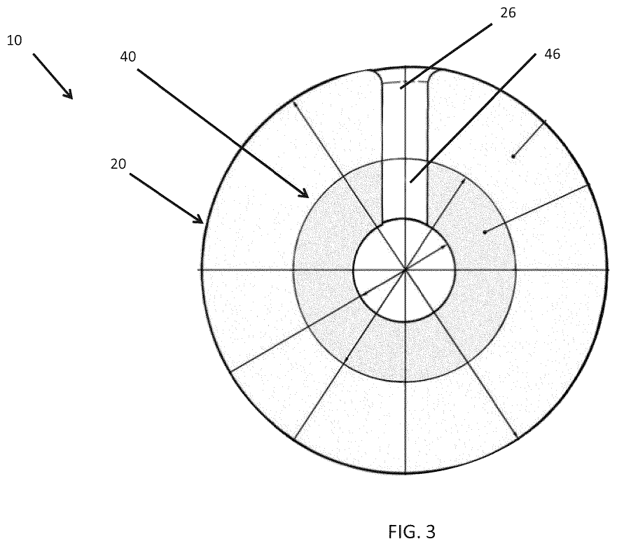

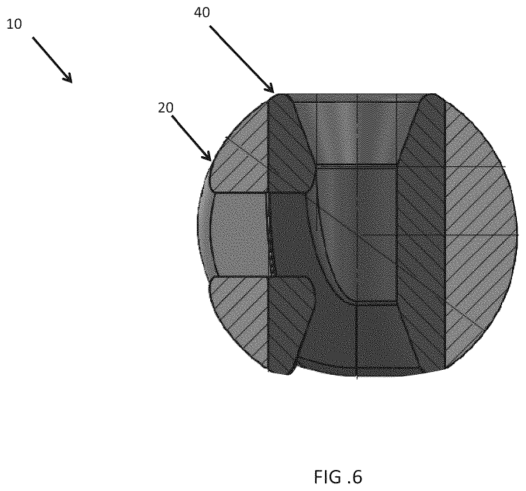

The first component 20 can also have a central or first aperture that is defined by the inner surface 30. The inner surface 30 can be cylindrical or at least partially cylindrical. In some examples, the central aperture defined by the inner surface 30 is a cylindrical or partially cylindrical aperture. As shown in FIG. 3, which illustrates a cross-sectional view along the width of the extremity offloading system 10, the central aperture can be a circle. In other examples, the central aperture can have different shapes, such as square, rectangular, triangular or another rounded shape (e.g. oval), in the cross sectional view along the width of the extremity offloading system 10. The central aperture can extend from the first end to the second end along the length of the first component 20. As shown in FIG. 6, which illustrates a cross-sectional view along the length of the extremity offloading system 10, the central aperture of the first component 20 can be rectangular in the cross sectional view. The diameter of the central aperture can be constant along the length of the first component 20. The central aperture defined by the inner surface 30 can receive the second component 40.

In some examples, the length between the first end and the second end of the first component 20 can be between 5 to 10 inches, such as between 5 to 6 inches, 6 to 7 inches, 7 to 8 inches, 8 to 9 inches, or 9 to 10 inches. In some examples, the outer diameter or width of the first component 20 can be between 5 to 15 inches, such as between 5 to 7 inches, 7 to 9 inches, 9 to 11 inches, 11 to 13 inches, or 13 to 15 inches. In some examples, the diameter of the central aperture of the first component 20 can be between 5 to 10 inches, such as between 5 to 6 inches, 6 to 7 inches, 7 to 8 inches, 8 to 9 inches, or 9 to 10 inches. It should be understood that the dimensions are not limited as such and the lengths and/or diameters may be lesser or greater than the disclosed examples.

The second component 40 can be an inner component that is configured to be within the first component 20. For example, the second component 40 can be received within the central aperture defined by the inner surface 30 of the first component 20. In some examples, the first component 20 and the second component 40 can be separate components, which can advantageously allow adjustment or replacement of individual components. In some examples, the first component 20 and the second component 40 can be bonded together. In some examples, the first component 20 and the second component 40 can be integral, which can allow for ease of use and transportation.

The second component 40 can be configured to receive at least a portion of a patient's leg, such as a patient's ankle or lower leg. The second component 40 can have an outer surface 48 that is cylindrical or at least partially cylindrical. The second component 40 can have a second thickness between the inner surface 50 and the outer surface 48.

The second component 40 can have an inner surface 50 that is cylindrical or at least partially cylindrical. The inner surface 50 can be configured to wrap around or partially wrap around a portion of a patient's leg or ankle. The inner surface 50 can define a central aperture configured to receive a portion of a patient's leg or ankle.

In some examples, the central aperture defined by the inner surface 50 is a cylindrical or partially cylindrical aperture. As shown in FIG. 3, which illustrates a cross-sectional view along the width of the extremity offloading system 10, the central aperture of the second component 40 can be a circle. In other examples, the central aperture can have different shapes, such as a partially rounded shape or an oval shape, in the cross sectional view along the width of the extremity offloading system 10. The central aperture can extend from the first end to the second end along the length of the second component 40. As shown in FIG. 6, which illustrates a cross-sectional view along the length of the extremity offloading system 10, a portion of the central aperture of the second component 40 can be rectangular in the cross sectional view. The first end and the second end of the central aperture of the second component 40 can be angled, such that the diameter increase towards the first end and the second end of the second component 40. As shown in FIG. 6, the cross-sectional view of the first end and second end of the central aperture of the second component 40 can be sloped or triangular in the cross-sectional view. For example, in some examples, the diameter of the central aperture of the second component 40 can be a first diameter in the center of the second component. The diameter of the central aperture towards the first end and the second end can be a second diameter, the second diameter greater than the first diameter. The diameter of the central aperture can gradually increase from the first diameter at the center to the second diameter at the first end or at the second end, such that the inner surface 50 can be sloped as the diameter increases.

In some examples, the length between the first end and the second end of the second component 40 can be between 5 to 10 inches, such as between 5 to 6 inches, 6 to 7 inches, 7 to 8 inches, 8 to 9 inches, or 9 to 10 inches. In some examples, the diameter of the second component 40 can be between 5 to 15 inches, such as between 5 to 7 inches, 7 to 9 inches, 9 to 11 inches, 11 to 13 inches, or 13 to 15 inches. In some examples, the first diameter of the central aperture of the second component 40 can be between 1 to 5 inches, such as between 1 to 2 inches, 2 to 3 inches, 3 to 4 inches, or 4 to 5 inches. In some examples, the second diameter of the central aperture of the second component 40 can be between 5 to 10 inches, such as between 5 to 6 inches, 6 to 7 inches, 7 to 8 inches, 8 to 9 inches, or 9 to 10 inches. It should be understood that the dimensions are not limited as such and the lengths and/or diameters may be lesser or greater than the disclosed examples.

The first component 20 and the second component 40 can be constructed from any material, such as foam. In some examples, the first component 20 and the second component 40 can be made of the same material or different material. In some examples, the first component 20 can be made of a material that has an increased firmness compared to the material of the second component 40. In some examples, the second component 40 can made of memory foam. The reduced firmness of the second component 40 can be provided for the comfort of the patient and to reduce the risk of irritation, as the second component 40 can be in direct contact with the patient's leg. The increased firmness of the first component 20 can be provided to provide support to the patient's leg or ankle as the patient applies weight against the extremity offloading system 10, which may rest on a surface. In some examples, the first component 20 and/or the second component 40 can be made of an antimicrobial or antibacterial material. In some examples, the material of the first component 20 and/or the second component may include holes or may be made of a breathable material for the patient's comfort and to prevent infection. In some cases, the first component 20 and/or the second component 40 can be coated in a layer of an antimicrobial or antibacterial material. In some examples, the first component 20 and/or the second component 40 can be made of a material that is latex free, which advantageously makes the extremity offloading system 10 biocompatible. In some examples, the first component 20 and/or the second component 40 can be made of or coated with a water repellent material or coating. Advantageously, forming the components 20 and 40 with or coating the components with water repellant material enables the extremity offloading system 10 to be made wet, such as from perspiration or water, or other bathing solution, without damaging the extremity offloading system 10. In some examples, the first component 20 and/or the second component 40 can be made of a foam with a density between 1 lb/ft³ to 26 lb/ft³, and in some examples between 1 lb/ft³ to 10 lb/ft³ or between 3 lb/ft³ to 6 lb/ft³.

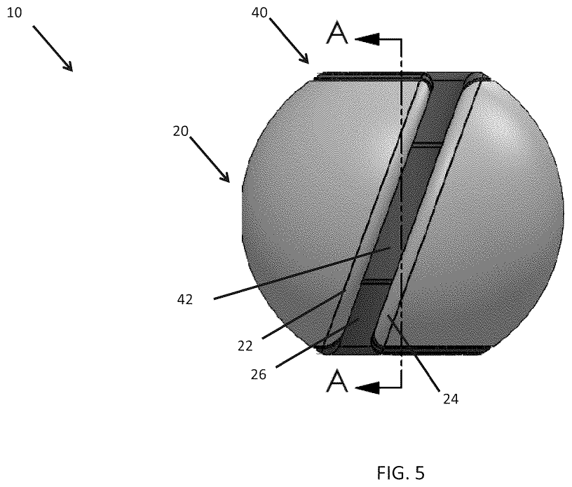

The first component 20 can have a longitudinal axis extending along the length of the first component 20 can extend between a proximal end and a distal end. As shown in FIG. 5, the first component 20 can have a first opening 26 that extends through the first thickness of the first component 20 on the front or anterior side of the first component 20. The first opening 26 can extend from the outer surface 28 to the inner surface 30. In this manner, the opening 26 provides access to the central aperture defined by the inner surface 30. In this manner, the outer surface 28 and the inner surface 30 are discontinuous along the circumference of the extremity offloading system 10. The opening 26 can extend from the proximal end to the distal end of the first component 20. The first opening 26 can be straight, such that it is substantially parallel to the longitudinal axis of the first component 20. As illustrated in FIG. 5, the opening 26 can be slanted or angled, such that it is slanted or angled relative to the longitudinal axis of the first component 20. In some embodiments, the slant or angle of the opening can be 5 degrees, 10 degrees, 15 degrees, 20 degrees, 25 degrees, 30 degrees, 35 degrees, 40 degrees, or 45 degrees, or any angle in between the foregoing or lesser or greater than the foregoing. The first component 20 can have a first edge 22 and a second edge 24 wherein the space between the first edge 22 and the second edge 24 define the first opening 26. The first edge 22 can be considered a medial edge. The second edge 24 can be considered a lateral edge. In some examples, the medial edge and the lateral edge may be reversed. The first edge 22 can be parallel to the second edge 24. The first edge 22 can be separated from the second edge 24 to define the first opening 26. In some examples, the first edge 22 and the second edge 24 can each be beveled. The beveled edges can advantageously allow the first component 20 and the second component 40 to remain secured to the patient. In some examples, the first edge 22 and the second edge 24 can each be straight edges.

Similarly, the second component 40 can have a longitudinal axis extending along the length of the second component 40 and can extend from a bottom portion or end to a top portion or end. As shown, the second component 40 can be a cylinder with the proximal end and the distal end each being flat. The proximal end can be considered the top end. The distal end can be considered the bottom end. Furthermore, in some embodiments, the proximal end and distal end may be reversed. The second component 40 can have a second opening 46 that extends through the second thickness of the second component 40. The second opening 46 can extend from the outer surface 48 to the inner surface 50. In this manner, the opening 46 provides access to the central aperture defined by the inner surface 50. The opening 46 can extend from the proximal end to the distal end of the second component 40. The second opening 46 can be straight or curved. The opening 46 can be slanted or angled, such that it is slanted or angled relative to the longitudinal axis of the second component 40. In some embodiments, the slant or angle of the opening can be 5 degrees, 10 degrees, 15 degrees, 20 degrees, 25 degrees, 30 degrees, 35 degrees, 40 degrees, or 45 degrees, or any angle in between the foregoing or lesser or greater than the foregoing. The second component 40 can have a first edge 42 and a second edge 44. The first edge 42 can be considered a medial edge. The second edge 44 can be considered a lateral edge. In some examples, the medial edge and the lateral edge may be reversed. The first edge 42 can be parallel to the second edge 44. The first edge 42 can be separated from the second edge 44 to define the first opening 26.

The first opening 26 of the first component 20 can match or substantially match the second opening 46 of the second component 40, such that the first opening 26 and the second opening 46 are aligned when the second component 40 is inserted into the first component 20. In this way, the first opening 26 and the second opening 46 can be configured to open and close together when the extremity offloading system 10 is being handled, applied, or repositioned.

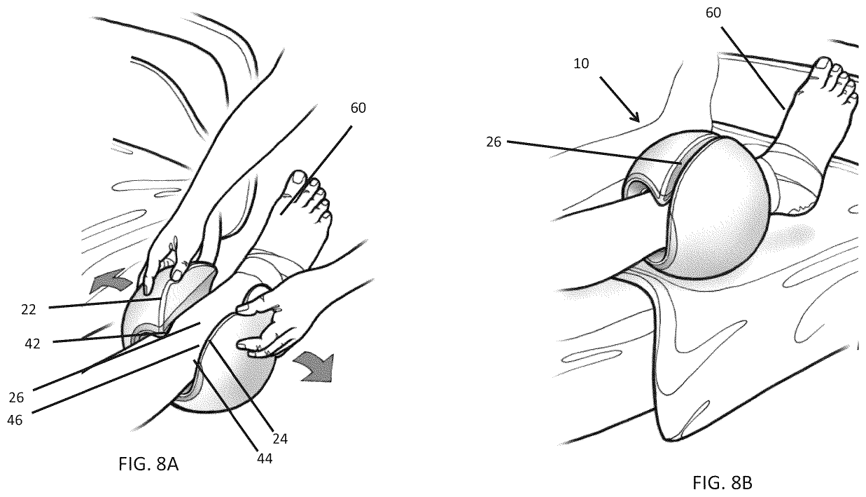

The extremity offloading system 10 can have a clamshell opening that can be configured to receive a portion of a leg or ankle 60 of a patient. As shown in FIG. 8A, in the open position, the extremity offloading system 10 can be opened to receive the portion of a leg or ankle 60. In the open position, the first edge 22 and the second edge 24 of the first component can be in widened or separated. The first edge 22 and the second edge 24 of the first component 20 can be separated to widen or increase the size of first opening 26. Similarly, the first edge 42 and the second edge 44 of the second component 40 can be separated to widen or increase the size of the second opening 46. The material of the first component 20 and the second component 40 can be flexible enough to allow the openings 26, 46 to be opened and be resilient enough to close the openings 26, 46 to secure the extremity offloading system 10 to the patient. The openings 26, 46 may automatically close when released by a user, or may be manually closed by the user. In some embodiments, the first opening 26 and second opening 46 can be configured to open and close together. In some embodiments, the first opening 26 and the second opening 46 can be configured to open and close independently from one another.

As the patient's leg or ankle 60 is inserted through the first opening 26 and the second opening 46, the patient's leg or ankle 60 may then be inserted in the space or aperture of the extremity offloading system 10. As described above, the space or aperture may be defined by the inner surface 50 of the second component 20. The inner surface 50 of the second component 20 can be configured to at least partially wrap around the portion of the patient's leg or ankle 60. The patient's leg or ankle 60 being inserted to the central space or aperture of the extremity offloading system 10 can cause closure of the extremity offloading system 10. This advantageously allows the extremity offloading system 10 to secure to the patient's leg or ankle 60 without the use of straps or other closure components. Without the use of straps or other closure components, the chances of error in installation or application of the extremity offloading system 10 is reduced, which can increase patient compliance and reduce injuries, including reducing injuries related to or caused by application of a device. The design can also allow for intuitive application and removal. The ease of application and securement can be done by one healthcare worker as opposed to some other systems which may require multiple people to apply. This can also reduce the need for a caretaker to continually monitor or adjust the extremity offloading system 10 on the patient.

Furthermore, the gravity or weight of the patient's leg or ankle 60 within the central aperture can not only close the extremity offloading system 10, but the gravity or weight of the patient's leg or ankle 60 can keep the extremity offloading system 10 closed. This advantageously allows the extremity offloading system 10 to be positioned on and retained on a patient's leg or ankle 60 without the use of fasteners or straps, thus making it easier and more convenient to position on/around and/or remove from an extremity of the patient. Further, the improved ease of using or positioning and removing the extremity offloading system 10 may result in improved patient compliance with maintaining use and positioning of the extremity offloading system 10. The overall spherical shape of the extremity offloading system 10 and curvature of the outer surface 28 advantageously allows for gravity to maintain the device in its proper position.

Additionally, the slanted openings along the length of the extremity offloading system 10 allows for easy application of the device for ease and comfort in application and to prevent inadvertent removal of the extremity offloading system 10.

As shown in FIG. 8B, in the closed position, the extremity offloading system 10 can surround a portion of the patient's leg or ankle 60. In some examples, the extremity offloading system 10 can be used in patients at risk for DTPI. In some examples, the extremity offloading system 10 can also be used on an amputated limb or extremity, such as on a leg with a below knee amputation. Once a limb is amputated, a patient often puts excess pressure on the opposite limb, for example in order to shift in bed. This increased pressure on the non-amputated limb can subsequently develop into a DTPI. As described above, for patients after a first amputation, approximately 30% to 50% of those patients will require an amputation of the opposite limb within 3 to 5 years. Use of the extremity offloading system 10 can prevent a DTPI from developing in the heel of the non-amputated limb and prevent or reduce the risk of requiring an amputation of the non-amputated limb. The extremity offloading system 10 can thus be used on either or both of the amputated limb and non-amputated limb. In the closed position, the first edge 22 and the second edge 24 of the first component 20 can be in close proximity or substantially closed. Similarly, the first edge 42 and the second edge 44 of the second component 40 can be in close proximity or substantially closed. In some examples, the first edge 22 and the second edge 24 can be in contact with one another in the closed position. Similarly, the first edge 42 and the second edge 44 of the second component 40 can be in contact with one another in the closed position. In some examples, the first edge 22 and the second edge 24 of the first component 20 as well as the first edge 42 and the second edge 44 of the second component 40 can be minimally separated. For example, the first opening 26 can be between 0.5 mm to 2 mm, such as between 0.5 mm to 1 mm, 1 mm to 1.5 mm, or 1.5 mm to 2 mm in the closed position. It should be understood that the dimensions are not limited as such and the first opening 26 can be lesser or greater than the disclosed examples.

As shown in FIG. 8B, when the extremity offloading system 10 is secured to the patient's leg or ankle 60 and the patient's leg is resting substantially parallel to a surface, the extremity offloading system 10 elevates or suspends the patient's heel, thus reducing the weight applied to the back of a patient's heel.

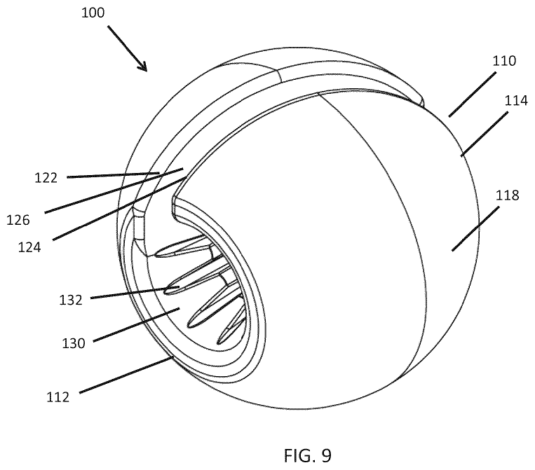

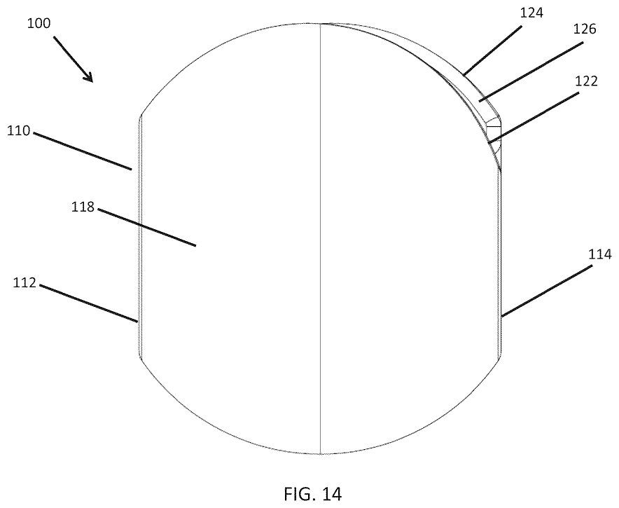

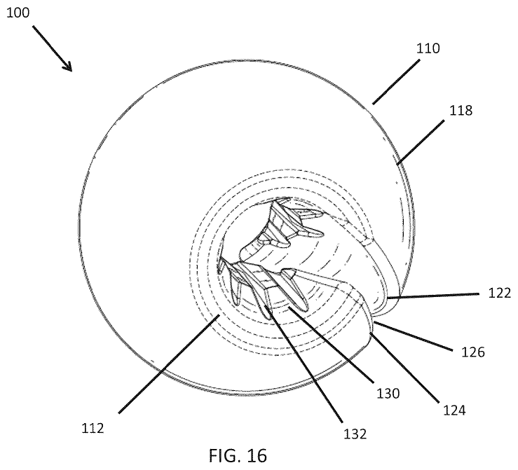

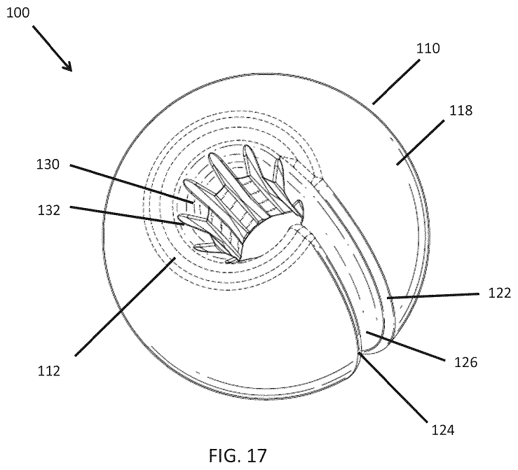

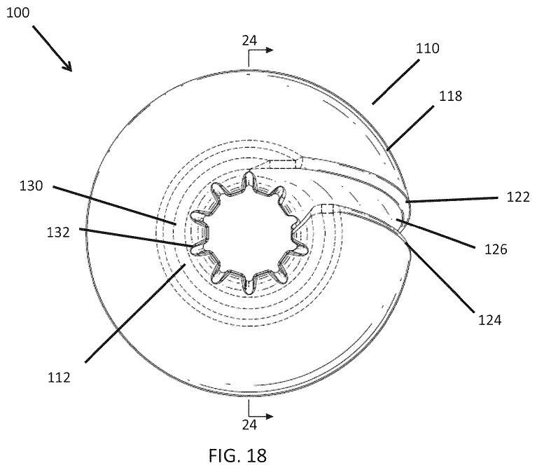

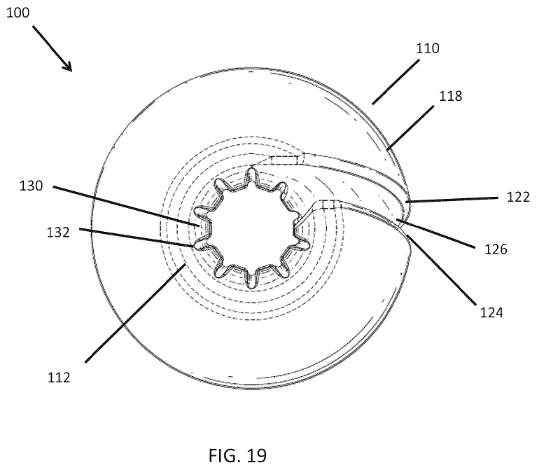

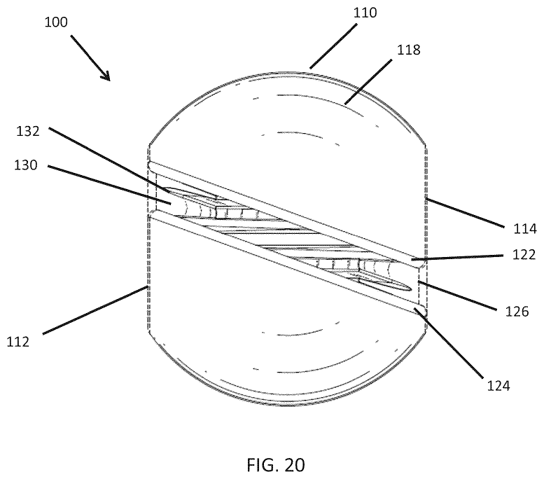

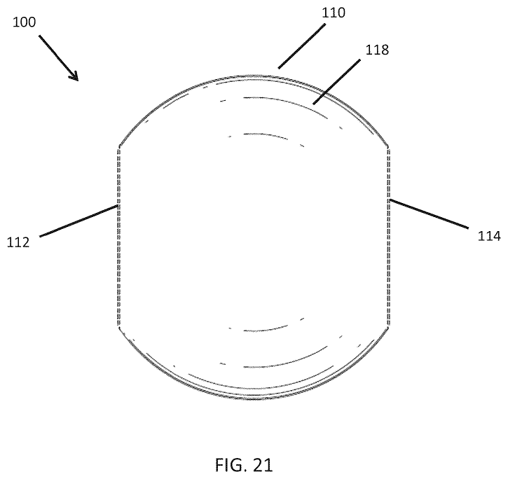

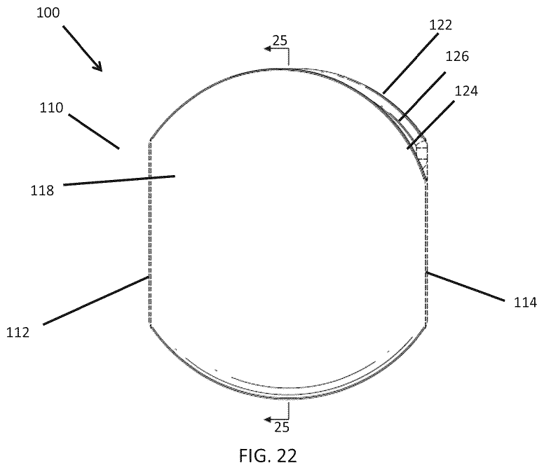

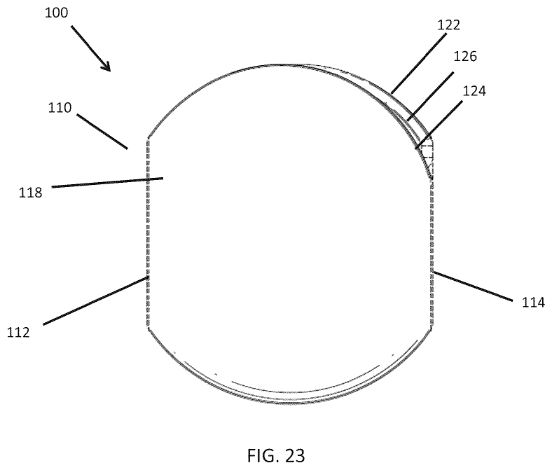

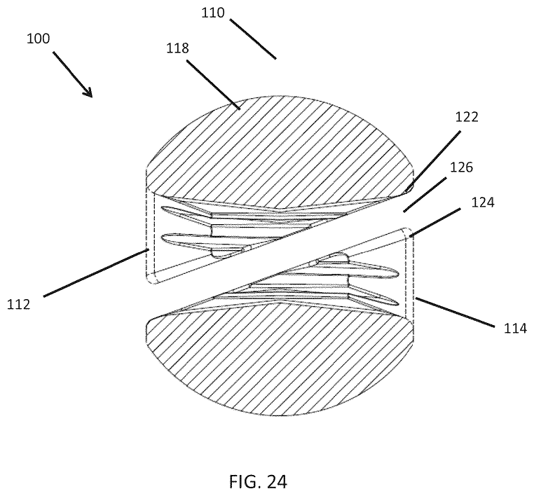

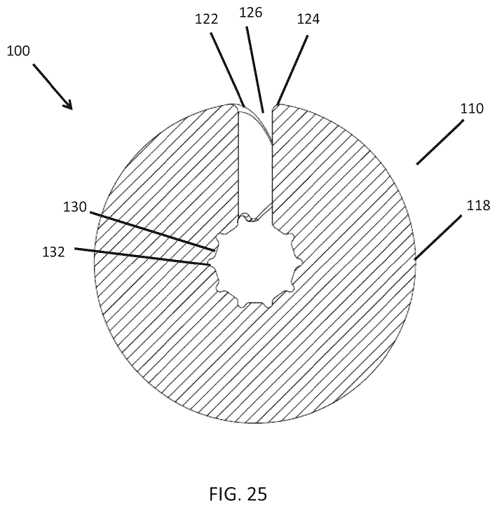

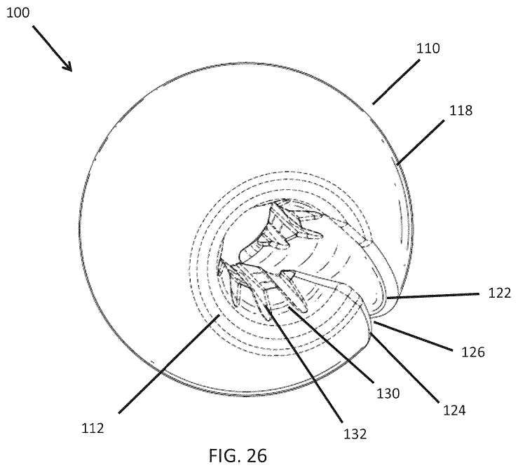

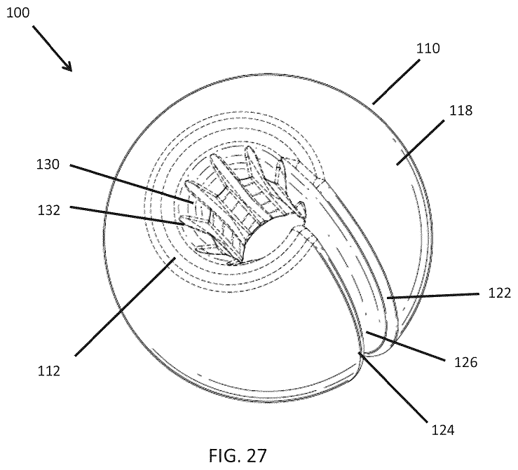

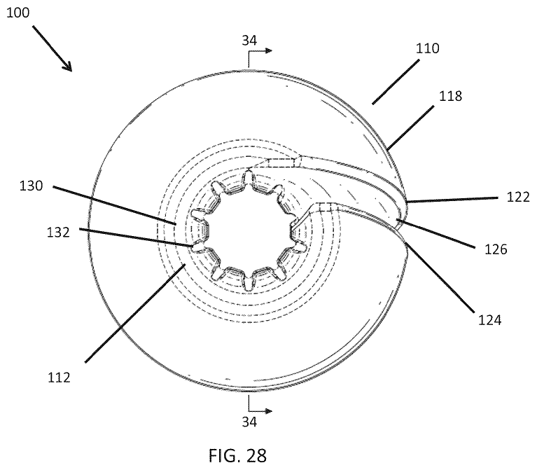

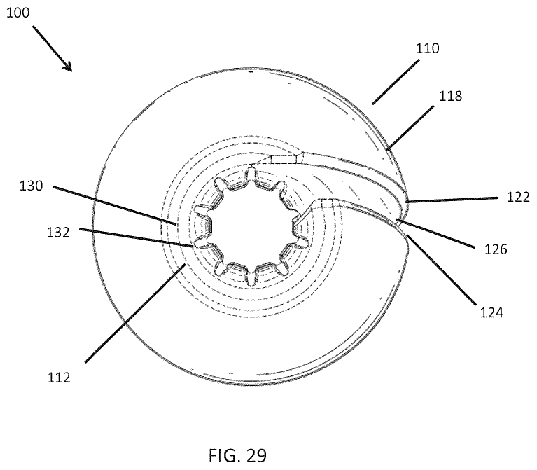

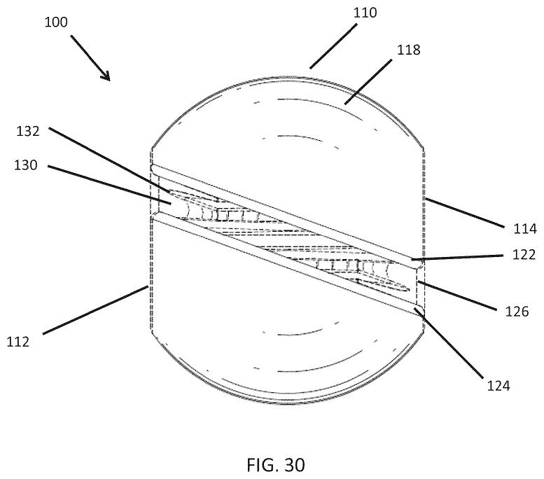

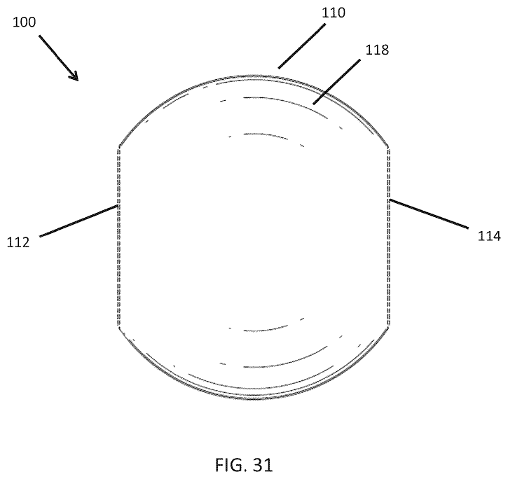

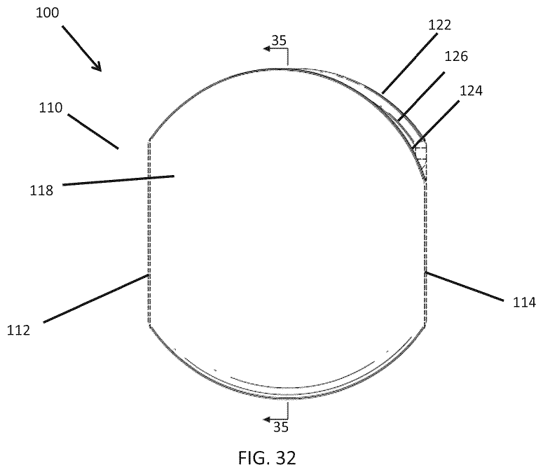

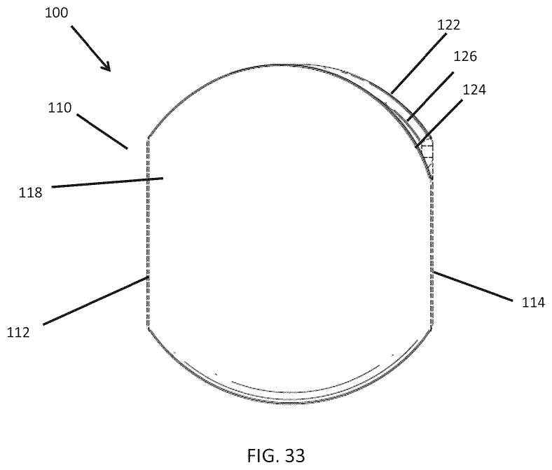

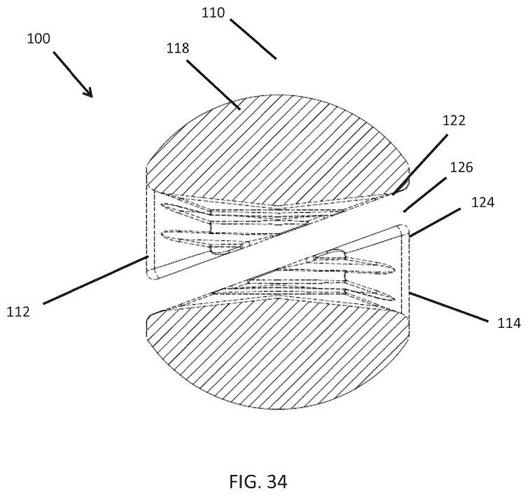

As shown in FIGS. 9-14 and 16-35, the extremity offloading system 100 can include a body 110, which can have a spherical shape. The extremity offloading system 100 can also be a unitary structure, as in one-piece. Similar to the extremity offloading system 10, the extremity offloading system 100 can also be called a support system, a support assembly, a suspension system, a suspension assembly or a sphere for an extremity, limb, or heel. The body 110 can have an outer surface 118 that is spherical or round or at least partially spherical or round. As shown, the proximal end 112 and the distal end 114 can each be flat, such that the body 110 does not form a full sphere. The proximal end 112 can be considered the top end. The distal end 114 can be considered the bottom end. Furthermore, in some embodiments, the proximal end 112 and distal end 114 may be reversed. The body 110 can have a thickness between the inner surface 130 and the outer surface 118.

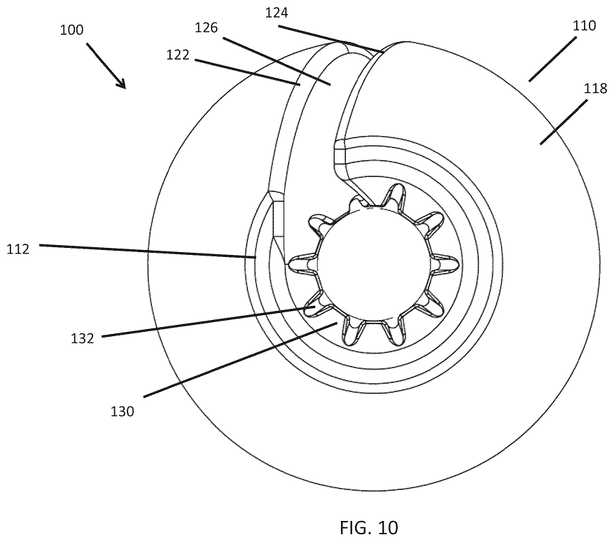

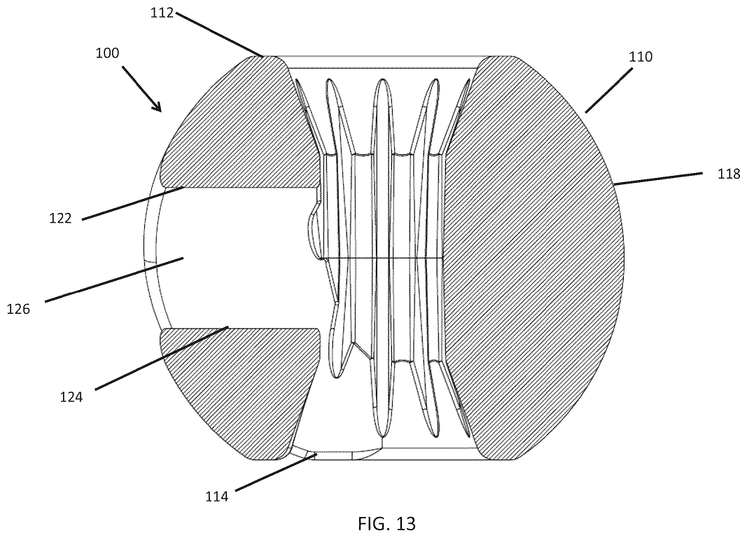

The body 110 can also have a central or first aperture that is defined by the inner surface 130. The inner surface 130 can be cylindrical or at least partially cylindrical. In some examples, the central aperture defined by the inner surface 130 is a cylindrical or partially cylindrical aperture. As shown in FIG. 10, which illustrates a top or end view of the extremity offloading system 100, the central aperture can be a circle. In other examples, the central aperture can have different shapes, such as square, rectangular, triangular or another rounded shape (e.g. oval). The central aperture can extend from the first end to the second end along the length of the body 110, such as from the proximal end 112 to the distal end 114. As shown in FIG. 13, which illustrates a cross-sectional view along the length of the extremity offloading system 100, the central aperture of the body 110 can be rectangular in the cross sectional view at least along a portion of the length of the body 110. The first end and the second end of the central aperture of body 110 be angled, such that the diameter increases towards the proximal end 112 and the distal end 114 of the body 110. As shown in FIG. 13, the cross-sectional view of the central aperture of the body 110 can have a first end and a second end which are sloped or triangular in the cross-sectional view. For example, in some examples, the diameter of the central aperture of the body 110 can be a first diameter in the center or middle of the body 110. The diameter of the central aperture can increase towards the first end and the second end to a larger, second diameter. The diameter of the central aperture can gradually increase from the first diameter at the center to the second diameter at the first end or at the second end, such that the inner surface 130 can be sloped as the diameter increases.

The central aperture defined by the inner surface 130 which can receive the patient's leg or ankle. The central aperture can taper outward at the end, such that the diameter of the central aperture increases towards the proximal end 112 and the distal end 114 of the body 110. This can advantageously provide comfort to the patient when the extremity offloading system 100 is positioned on the patient. In other examples, the diameter of the central aperture can be constant along the length of the body 110.

In some examples, the length between the first end and the second end of the first component 20 can be between 2 to 10 inches, such as between 2 to 4 inches, 5 to 10 inches, such as between 5 to 6 inches, 6 to 7 inches, 7 to 8 inches, 8 to 9 inches, or 9 to 10 inches. In some examples, the outer diameter or width of the first component 20 can be between 5 to 15 inches, such as between 5 to 7 inches, 7 to 9 inches, 9 to 11 inches, 11 to 13 inches, or 13 to 15 inches. In some examples, the diameter of the central aperture of the body 120 can be between 5 to 15 inches, such as between 5 to 6 inches, 6 to 7 inches, 7 to 8 inches, 8 to 9 inches, 9 to 10 inches, 10 to 11 inches, 11 to 12 inches, 12 to 13 inches, or 14 to 15 inches. It should be understood that the dimensions are not limited as such and the lengths and/or diameters may be lesser or greater than the disclosed examples.

The body 110 can have a plurality of flutes or cutouts 132 along the inner surface 130 of the central aperture. Each of the plurality of flutes can have a length that extend along the length of the central aperture. The plurality of flutes 132 can extend along the circumference of the central aperture along the inner surface 130. The plurality of flutes 132 can be equally spaced around a circumference of the aperture defined by the inner surface 130. The plurality of flutes 132 are positioned along the inner surface 120 of the central aperture, such that each of the plurality of flutes are configured to be positioned against a patient's leg when the patient's leg is inserted in the central aperture. These flutes 132 can advantageously allow air to flow through the flutes 132 to provide comfort to the patient while the device is in use. For example, the flutes 132 allow air to flow which can prevent too much friction from occurring between the inner surface 130 and the patient's skin. The flutes 132 can also lower the temperature of the patient's leg with the device positioned thereon. The flutes 132 can also reduce moisture or humidity build up of the patient with the device positioned thereon. The body 110 can have any number of flutes, such as 6, 8, 10, 12, or 14 flutes. As shown, the body 110 can have 10 flutes, which can advantageously provide adequate air flow and support, while preventing the creation of too many pressure points for patient comfort.

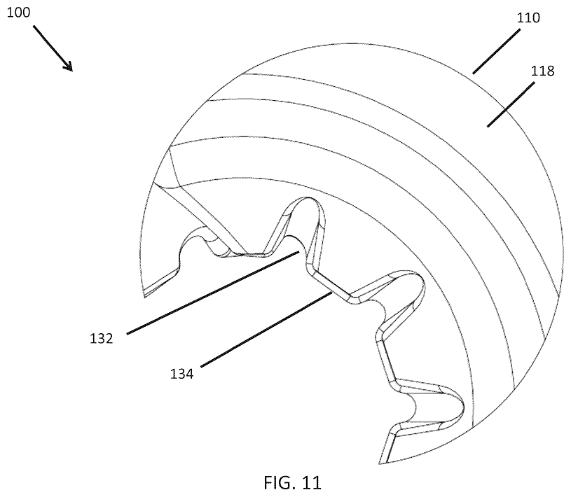

There can be a plurality of portions 134 of the inner surface 130 between each of the plurality of flutes 132. For example, as shown in FIG. 11, the plurality of portions 134 can be flat portions or surfaces. The plurality of portions 134 being flat can advantageously allow for a more uniform surface, comfortable contact with the patient's skin, and better contact for any surfaces positioned therein. In other examples, the plurality of portions 134 can be triangular or rounded or any other shape. In some examples, as shown in FIG. 11, the corners between each of the portions 134 and the flutes 132 may be rounded. In some examples, the corners between each of the portions 134 and the flutes 132 may be sharp.

In some examples, the depth of each flute 132, which can be measured radially from the inner surface 130, can be between 0.1 to 1 inches, such as between 0.1 to 0.2 inches, 0.2 to 0.3 inches, 0.3 to 0.4 inches, 0.5 to 0.6 inches, 0.6 to 0.7 inches, 0.7 to 0.8 inches, 0.8 to 0.9 inches, or 0.9 to 1 inches. In some examples, the width of each flute 132, which can be measured at a base of each flute 132, can be between 1 to 5 inches, such as between 1 to 2 inches, 2 to 3 inches, 3 to 4 inches, or 4 to 5 inches. In some examples, the width of the flat surface of the plurality of portions 134 can be between 0.1 to 1 inch, such as between 0.1 to 0.3 inches, 0.3 to 0.5 inches, 0.5 to 0.7 inches, or 0.7 to 0.9 inches. In some examples, the angle between each of the flutes 132 can be between 15° to 45°, such as between 20° to 30° or between 30° to 40°. In some examples, the angle between each of the plurality of portions 134 can be between 15° to 45°, such as between 20° to 30° or between 30° to 40°.

The body 120 can be constructed from any material, such as foam. In some examples, the body 120 can made of memory foam. In some examples, the body 120 can be made of an antimicrobial or antibacterial material. In some examples, the material of the body 120 may include holes or may be made of a breathable material for the patient's comfort and to prevent infection. In some examples, the body 120 can be coated in a layer of an antimicrobial or antibacterial material. In some examples, the body 120 can be made of a material that is latex free, which advantageously makes the extremity offloading system 100 biocompatible. In some examples, the body 120 can be made of or coated with a water repellent material or coating. In some examples, the body 120 can be made of a foam with a density between 1 lb/ft³ to 26 lb/ft³, and in some examples between 1 lb/ft³ to 10 lb/ft³, 3 lb/ft³ to 6 lb/ft³, or 3 lb/ft³ to 4 lb/ft³ Based on the clinical study, as discussed further below, different densities can be used based on the patient's BMI. Higher densities can be used for high patient BMIs. For example, a first version can use foam with a first density of 4 lb/ft³ (such as foam 274) for heavier BMIs (such as over 190 lbs or a BMI over 30) and a second version can use foam with a second density of 3 lb/ft³ (such as foam 266) for lighter BMIs (such as less than 190 lbs or BMI under 30). Furthermore, the foam can be a polyurethane foam. The use of foam can be advantageous in that it can allow the body 120 to be compressed and vacuum packed. This can reduce the required storage space and increase the accessibility of the system, which can in turn encourage healthcare providers to utilize the device, increasing use and compliance.

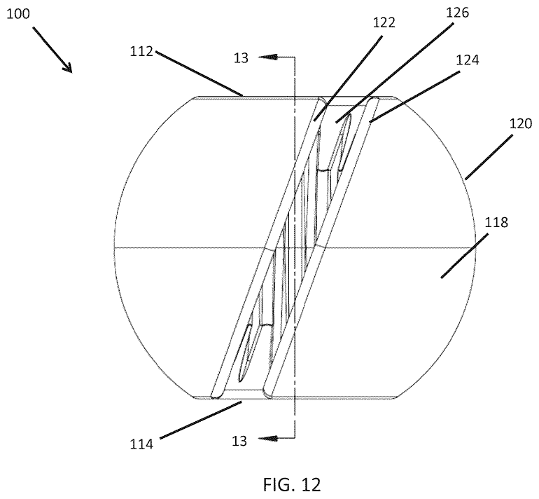

The body 110 can have a longitudinal axis extending along the length of the body 120, which can extend between a proximal end and a distal end. As shown in FIG. 12, the body 110 can have an opening 126 that extends through a side of the body 110, such that the opening 126 extends through the thickness of the body 110 on the front or anterior side of the body 110. The opening can extend from the outer surface 118 to the inner surface 130. In this manner, the opening 126 provides access to the central aperture defined by the inner surface 130. In this manner, the outer surface 118 and the inner surface 130 are discontinuous along the circumference of the extremity offloading system 100. The opening 126 can extend from the proximal end 112 to the distal end 114 of the body 110. The opening 126 can be straight, such that it is substantially parallel to the longitudinal axis of the body 110. As illustrated in FIG. 12, the opening 26 can be slanted or angled, such that it is slanted or angled relative to the longitudinal axis of the body 110. In some embodiments, the slant or angle of the opening can be 5 degrees, 10 degrees, 15 degrees, 20 degrees, 25 degrees, 30 degrees, 35 degrees, 40 degrees, or 45 degrees, or any angle in between the foregoing or lesser or greater than the foregoing. The body 110 can have a first edge 122 and a second edge 124 wherein the space between the first edge 122 and the second edge 124 define the opening 126. The first edge 122 can be considered a medial edge. The second edge 124 can be considered a lateral edge. In some examples, the medial edge and the lateral edge may be reversed. The first edge 122 can be parallel to the second edge 124. The first edge 122 can be separated from the second edge 124 to define the first opening 26. The first edge 122 can be evenly spaced from the second edge 124. In some examples, the first edge 122 and the second edge 124 can each be beveled. The beveled edges can advantageously allow the body 110 to remain secured to the patient. In some examples, the first edge 122 and the second edge 124 can each be straight edges. In some examples, the width of the opening 126 can be between 0.5 inches to 2 inches, such as between 0.5 inches to 1 inch, between 1 to 1.5 inches, between 1.5 inches to 2 inches.

The extremity offloading system 100 can be configured to receive a portion of a leg or ankle of a patient. Similar to extremity offloading system 10 as shown in FIG. 8A, in the open position, the extremity offloading system 100 can be opened to receive the portion of a leg or ankle. In the open position, the first edge 122 and the second edge 124 of the body 110 can be in widened or separated. The first edge 122 and the second edge 124 of the body 110 can be separated to widen or increase the size of the opening 126. The material of the body 110 can be flexible enough to allow the opening 126 to be opened and be resilient enough to close the opening 126 to secure the extremity offloading system 100 to the patient. The opening 126 may automatically close when released by a user, or may be manually closed by the user.

As the patient's leg or ankle is inserted through the opening 126, the patient's leg or ankle may then be inserted in the space or aperture of the extremity offloading system 100. As described above, the space or aperture may be defined by the inner surface 130 of the body 110. The inner surface 130 of the body 110 can be configured to at least partially wrap around the portion of the patient's leg or ankle. The patient's leg or ankle being inserted to the central space or aperture of the extremity offloading system 100 can cause closure of the extremity offloading system 100. This advantageously allows the extremity offloading system 100 to secure to the patient's leg or ankle without the use of straps or other closure components. Without the use of straps or other closure components, the chances of error in installation or application of the extremity offloading system 100 is reduced, which can increase patient compliance and reduce injuries. The ease of application and securement can be done by one healthcare worker as opposed to some other systems which may require multiple people to apply. This can also reduce the need for a caretaker to continually monitor or adjust the extremity offloading system 100 on the patient. The ease of application and securement of the system 100 can also advantageously allow a single healthcare worker to apply medicine or other treatments, such as a wound vac, to the area or adjacent areas. These treatment regimens can also advantageously be executed without removing the system 100, as the limb is already offloaded, raised, and can be easily exposed or accessed. Furthermore, the system 100 can also advantageously prevent injury not only to the patient but to the healthcare worker, such as by avoiding the need to lift the patient's limb by the healthcare worker.

Furthermore, the gravity or weight of the patient's leg or ankle within the central aperture can not only close the extremity offloading system 100, but the gravity or weight of the patient's leg or ankle can keep the extremity offloading system 100 closed. This advantageously allows the extremity offloading system 100 to be positioned on and retained on a patient's leg or ankle without the use of fasteners or straps, thus making it easier and convenient for the patient and improve patient compliance. The overall spherical shape of the extremity offloading system 100 and curvature of the outer surface 118 advantageously allows for gravity to maintain the device in its proper position.

Additionally, the slanted opening 126 along the length of the extremity offloading system 100 allows for easier application of the device for ease and comfort in application and to prevent inadvertent removal of the extremity offloading system 100.

Similar to the extremity offloading system 10 as shown in FIG. 8B, the extremity offloading system 100 in the closed position can surround a portion of the patient's leg or ankle 60. In some examples, the extremity offloading system 100 can be used in patients at risk for DTPI. In some examples, the extremity offloading system 100 can also be used on an amputated limb or extremity, such as on a leg with a below knee amputation. Once a limb is amputated, a patient often puts excess pressure on the opposite limb, for example in order to shift in bed. This increased pressure on the non-amputated limb can subsequently develop into a DTPI. The extremity offloading system 100 can thus be used on either or both of the amputated limb and non-amputated limb. In the closed position, the first edge 122 and the second edge 124 of the body 110 can be in close proximity or substantially closed. In some examples, the first edge 122 and the second edge 124 can be in contact with one another in the closed position. In some examples, the first edge 122 and the second edge 124 of the body 110 can be minimally separated. For example, the opening 126 can be between 0.5 mm to 2 mm, such as between 0.5 mm to 1 mm, 1 mm to 1.5 mm, or 1.5 mm to 2 mm in the closed position. It should be understood that the dimensions are not limited as such and the opening 126 can be lesser or greater than the disclosed examples. When the extremity offloading system 100 is secured to the patient's leg or ankle and the patient's leg is resting substantially parallel to a surface, the extremity offloading system 100 elevates or suspends the patient's heel, thus reducing the weight applied to the back of a patient's heel.

Furthermore, the round outer surface 28, 118 of the first component 20 or the body 110 allows the patient to turn or rotate their leg or ankle 60, while still keeping the heel suspended. This advantageously allows the patient to move or shift while maintaining the desired function of the extremity offloading system 10, 100. Allowing movement also reduces the risk of an occurrence of DTPI to other parts of the body. Further, allowing movement of the limb can also prevent stagnation, thereby reducing chances of development of deep venous thrombosis or blood clots.

The compact size of the extremity offloading system 10, 100 also allows the extremity offloading system 10, 100 to be easily stored and to be easily transported. The compact size of the extremity offloading system 10, 100 also allows the patient's leg to be moved while wearing the extremity offloading system 10, 100.

Additionally, the extremity offloading system 10, 100 can be maintained and retained on the patient while not or minimally interfering with a patient's ability to walk or move. For example, a patient with the extremity offloading system 10, 100 on their leg or ankle can get up into a sitting position, a standing position, or even walk or shift without removing or adjusting extremity offloading system 10, 100. This can advantageously allow the extremity offloading system 10, 100 to more reliably support a user's heel or leg and providing adequate offloading by preventing pressure being applied to the area and ensuring the heels are not in contact with the support surface. The extremity offloading system 10, 100 can even continue to provide support as the user moves or shifts.

Another advantage of extremity offloading system 10, 100 is that the foot or heel of the patient is still accessible or exposed. Therefore, the heel or foot can still be treated or monitored without removing or adjusting the extremity offloading system 10, 100. For example, the extremity offloading system 10, 100 can allow for or facilitate contemporary methods of treatment of foot and ankle ulcerations like grafting and wound vac applications.

Yet another advantage of the extremity offloading system 10, 100 is that the assemblies can be used in various settings to position the foot or heel in the desired position, such as in a surgical operating room, for a procedure, or for in an imaging setting (such as x-rays).

Yet another advantage of the extremity offloading system 10, 100 is that the various components of the system can be made of antimicrobial or antibacterial material which can prevent or reduce the risk of contamination. The material can also advantageously allow for proper cleaning and disinfection of the system.

### Study

An IRB approved study was conducted in a community hospital, adhering to predefined patient selection criteria. The study involved 28 inpatients with data collected on multiple parameters over 72 hours between Jul. 24, 2023-Aug. 27, 2023. Participants were randomly assigned to receive and apply the extremity offloading system with two different material densities. Fourteen participants were given extremity offloading systems composed of Foam 266 and the remaining fourteen participants were given extremity offloading systems composed of Foam 274. Each extremity offloading system was uniquely identified and assigned based on a randomized order generated in the protocol. The extremity offloading systems were used continuously while patients were in bed at the hospital.

The primary aims of the study were to assess the effectiveness and consistency of offloading related to positioning and to evaluate the safety and comfort of the extremity offloading system. Patients were monitored over 72 hours to assess the effectiveness of the extremity offloading system. Effectiveness was assessed based on the criteria: (1) how well the device offloaded the heel and ankle, (2) how stationary the device was during use, e.g., was there movement of the device during use, and (3) how comfortable the device was during use for the patient.

Patients were instructed that they could remove the extremity offloading system 10, 100 when they were not in bed, such as when they were walking, eating, sitting up, and/or using the bathroom. As long as the patient was using the device while in bed, it was hypothesized that this would prevent the formation of HAPIs. Additional information was collected from each patient to further analyze the effectiveness of the extremity offloading system: (1) patient gender, assigned at birth, (2) BMI (normal weight, overweight, or obese), (3) device application (right or left), (4) device composition (foam 274 or foam 266).

Based on the data collected and evaluated by a statistician, the extremity offloading system proved suitable for use for offloading of the foot and ankle. The extremity offloading system statistically demonstrated effective heel offloading. In 93% of patients in the study, the device proved to be comfortable. Throughout the study, there were no reported skin conditions or medical device related tissue injuries as a direct result of the utilization of this device. Data collected from healthcare professionals indicated that the extremity offloading system was easy to use and provided effective offloading. Slight variation was seen between the two densities with reference to surface cracking.

Thus, the extremity offloading system used provides efficient offloading and can effectively used in the treatment and prevention of HAPIs. Based on the global design and rounded surfaces, the extremity offloading system allows for motion of the limb. The extremity offloading system can allow patients to turn in their beds without comprising offloading. The extremity offloading system can be strapless and thus reduces chances of the system causing related tissue injuries. The extremity offloading system can provide full heel offloading. The extremity offloading system can be lightweight, pleasant in design, easy to apply, and safe to apply.

The extremity offloading system can allow for easier healthcare access to the wounds. The extremity offloading system can be compatible with compressor devices. The extremity offloading system can not only be easily used with wound vacs, but also made the application of wound vacs easier and more effective. The extremity offloading system can also be easily utilized with other modalities, such as grafting. The extremity offloading system can facilitate easy observation to allow for healthcare professionals to observe wounds in the heel or ankle region. The extremity offloading system can also allow a single healthcare professional, such as one nurse, to evaluate the wound, without requiring additional assistance. The extremity offloading system can also prevent the need for a healthcare professional to lift the limb, which can in turn prevent other injuries from occurring to the patient or to the healthcare professional.

The extremity offloading system can have a sufficient length such that the central aperture or internal core of the extremity offloading system is long enough to support the limb without undue pressure. The shape and structure of the extremity offloading system can maintain the position without causing pressure on the limb. The length of the central aperture can be sufficient to distribute the weight over the length of the central aperture and thereby reduce medical device related tissue injury. The length of the central aperture can be maximized to allow the leg to rest in the offloading position and for a greater area for weight distribution thereby reducing potential points of stress of the system on the user's leg. The internal corridor may have flutes that allow for air flow and therefore provide reduced moisture.

The extremity offloading system can have sensor capabilities, as discussed further herein.

The extremity offloading system can be vacuum packaged allowing for easier accessibility and storage. For example, the extremity offloading system being partially or wholly made of foam can allow the extremity offloading system to be vacuum packaged. The extremity offloading system can also be compact and small compared to other devices, such as foot braces. The extremity offloading system can avoid the need for excess room which can allow the extremity offloading system to be easily stored and transported and therefore easily accessible for use. For example, one or more extremity offloading systems can be stored at nursing stations. This can allow the extremity offloading system to be used proactively to prevent injury or during early stages of potential problems.

### Offloading System Risers

In some cases, a patient's leg may be in contracture due, for example, to a patient injury. In other cases, it may be necessary to maintain the patient's leg in a position that may result in a state of contracture. Contracture can be classified into different levels of severity based on the degree of contracture. For example, a mild degree of contracture may include contracture of 1-19° while a contracture of between 20-40° may be considered a moderate degree of contracture. A contracture of greater than 40° may be considered severe. Other classifications of the degree of contracture may exist. Further, a severity of contracture may differ based on age, body type, and/or specific conditions of the patient.

Regardless of the reason that the patient's leg is in contracture, in some cases, an extremity offloading system, such as the extremity offloading system 100 may not provide sufficient support to offload the pressure applied to the patient's leg due to the contracture. For example, in cases where the degree of contracture exceeds an advanced mild degree of contracture (e.g., where the degree of contracture exceeds 10° of deformity or flexion of the knee) when the patient's leg is inserted in the body 110 of the extremity offloading system 100, the body 110 may not sufficiently offload the pressure applied to the patient's leg due to the existence of or the degree of contracture. Furthermore, when the patient's leg is inserted in the body 110, the patient's Achilles tendon can contract without further support and positioning.

In some embodiments, the extremity offloading system can include a riser or set of risers that can provide additional support and that can help position the extremity offloading system or the user's foot or leg in the extremity offloading system. The riser, which may also be referred to as a “raiser,” an “adjustment support,” or a “contraction adjustment support” herein, may be integrally formed with the extremity offloading system or may be a separate component that can be removably and/or permanently attached to the extremity offloading system. The use of the additional supports or risers can further position the body 110 or further position the patient's leg to provide additional support and reduce the pressure applied to the patient's leg. In some embodiments, the additional support provided by the contraction adjustment support can be beneficial in helping to alleviate pressure at least in cases of advanced mild to moderate contractures (e.g., between 10° to 40° of contracture), and in some cases even in severe cases of contracture (e.g., those exceeding 40°).

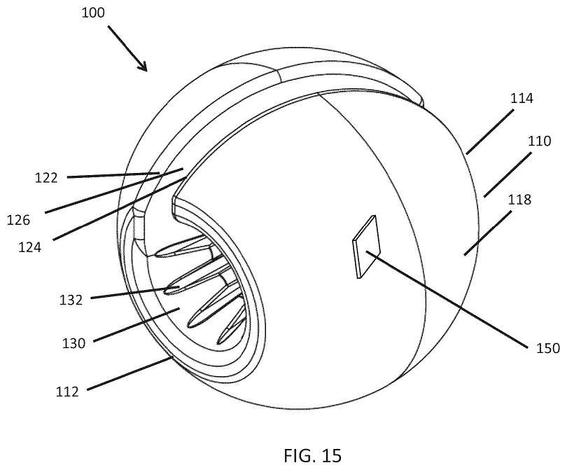

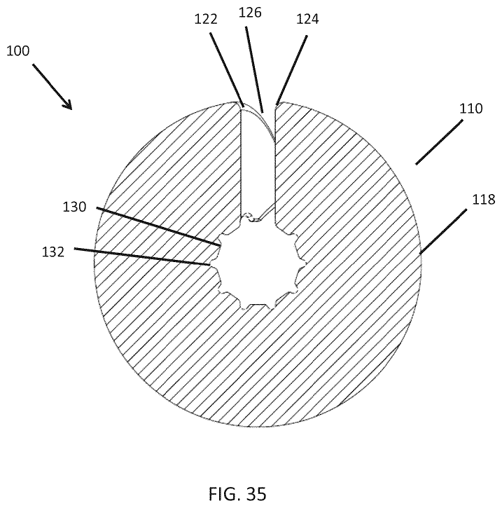

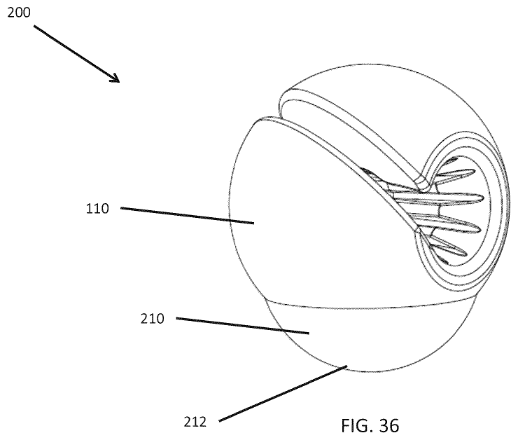

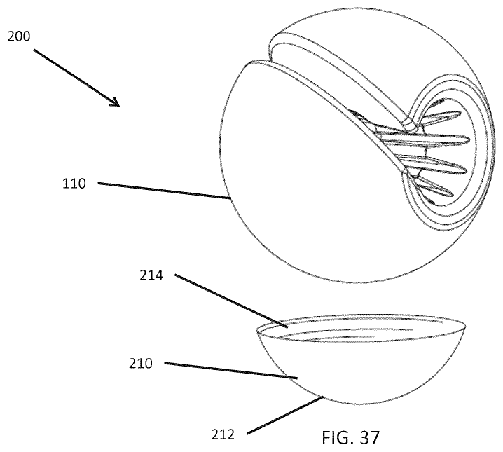

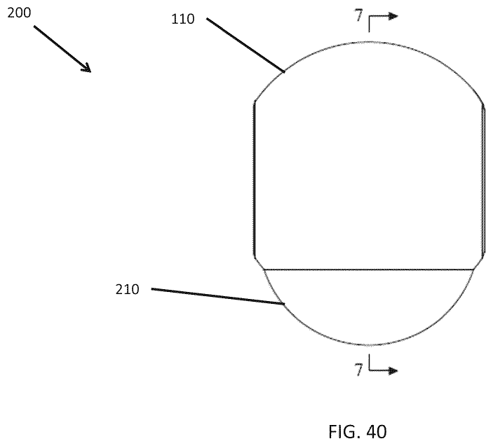

As shown in FIGS. 36-41, the extremity offloading system 200 can include a contraction adjustment support or raiser 210. In FIGS. 36-41, like numbers are used to refer to parts similar to those of FIGS. 9-35 and reference can be made to the description of those parts made with reference to FIGS. 9-35. As with the embodiments described with reference to FIGS. 9-35 the extremity offloading system 200 can include a body, which can have a substantially spherical shape. In the illustrated embodiments, the extremity offloading system 200 may be configured similarly to the extremity offloading system 100 of FIGS. 9-35. Additionally, the extremity offloading system 200 of FIGS. 36-41 can include a contraction adjustment support or raiser 210. The contraction adjustment support or raiser 210 can elevate or raise the position of the body 110 and thereby the patient's leg when inserted into the body 110. The contraction adjustment support 210 may be configured to be positioned on a surface beneath the patient, such as a bed. The contraction adjustment support 210 may be configured to be positioned between the surface it rests on and the body 110. The rounded bottom surface of the contraction adjustment support 210 can be positioned on the surface that the contraction adjustment support 210 rests on. The use of the contraction adjustment support 210 can further elevate the patient's leg, which can provide additional offloading of the patient's heel. This is particularly advantageous where a patient is experiencing contracture at the knee such that the body 110 cannot sufficiently offload pressure from the patient's heel and the patient's heel may be pushed down with the patient's weight even with use of the body 110. The additional contraction adjustment support 210 can counteract this additional weight and pressure on the patient's heel by further elevating the patient's heel.

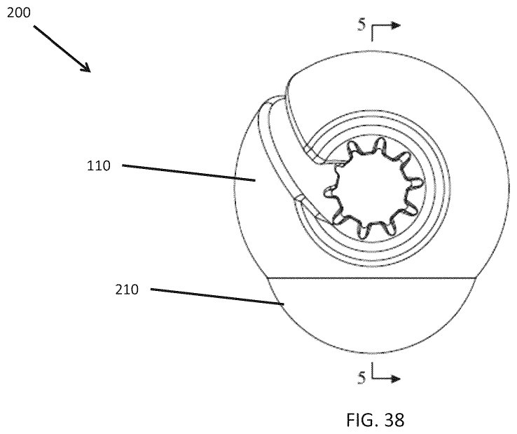

The contraction adjustment support 210 can have a bottom surface 212 that is spherical or round, or at least partially spherical or round. The rounded bottom surface 212 of the contraction adjustment support 210 enables the contraction adjustment support 210 to raise the extremity offloading system 200 while still permitting the extremity offloading system 200 to rotate with the support 210. The bottom surface 212 can be convex. For example, the extremity offloading system 200 can still rotate from side to side and/or front to back. In a side view, such as shown in FIG. 38, the contraction adjustment support 210 can have an overall semispherical shape. In a top view, the contraction adjustment support 210 can have a circular shape.

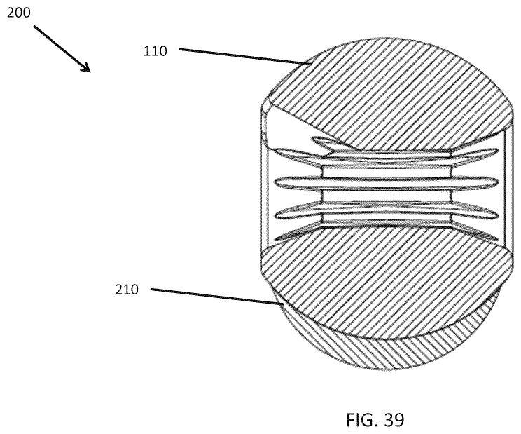

The contraction adjustment support 210 can have a top surface 214 that is concave. The top surface 214 can be spherical or round, or at least partially spherical or round. The concave top surface 214 can be configured to receive the bottom rounded surface of the body 110. In some examples, the concave top surface 214 of the contraction adjustment support 210 can have a curvature that substantially matches the curvature of the bottom surface of the body 110. The concave top surface 214 can include an adhesive that secure the body 110 and the contraction adjustment support 210 together. In this manner, the body 110 and the contraction adjustment support 210 can be removably attached. In some examples, the adhesive can be an adhesive tape, glue, hook and loop fasteners, magnetic fasteners, or snap fasteners. In a sectional view, such as shown in FIG. 39 or FIG. 41, the contraction adjustment support 210 can have a curved shape, such as a u-shape.

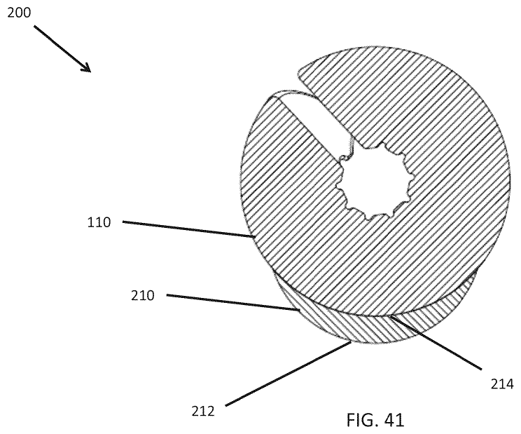

In some examples, as described above, the contraction adjustment support 210 can be separable from the body 110. In other examples, the contraction adjustment support 210 can be unitary with the body 110. In some examples, the contraction adjustment support 210 can come in a variety of sizes, such as to accommodate different sizes of patients or different levels of offloading. For example, the contraction adjustment support 210 can come in a variety of different heights. In other examples, the contraction adjustment support 210 can be one size. In some examples, rather than one contraction adjustment support 210 being used, two or more contraction adjustment supports 210 can be used. For example, as shown in FIGS. 42 and 43, two contraction adjustment supports 210 can be used adjacent to one another on a bottom surface of the body 110. This can be advantageous in cases where it is desirable to limit movement or rotation of the user's leg, such as in severe contracture cases or with patients who are unable to comply with maintaining a position of their leg without assistance.

In some examples, the contraction adjustment support 210 can be constructed from any material, such as foam. In some examples, the contraction adjustment support 210 can be made of memory foam. In some examples, the contraction adjustment support 210 can made of memory foam. In some examples, the contraction adjustment support 210 can be made of an antimicrobial or antibacterial material. In some examples, the material of the contraction adjustment support 210 may include holes or may be made of a breathable material for the patient's comfort and to prevent infection. In some examples, the contraction adjustment support 210 can be coated in a layer of an antimicrobial or antibacterial material. In some examples, the contraction adjustment support 210 can be made of a material that is latex free. In some cases, the extremity offloading system 200 may be biocompatible. In some examples, the contraction adjustment support 210 can be made of or coated with a water repellent material or coating. In some examples, the contraction adjustment support 210 can be made of a foam with a density between 1 lb/ft³ to 26 lb/ft³, and in some examples the density may be between 1 lb/ft³ to 10 lb/ft³ or 3 lb/ft³ to 6 lb/ft³. In some examples, the contraction adjustment support 210 can be made of the same or similar material as the body 110. In some examples, the foot support 310 can be made with a foam of a density between 3 lb/ft³ to 4 lb/ft³. Similar to the systems 10, 100 described above, the density of the contraction adjustment support 210 can be based on the patient's BMI. Higher densities can be used for high patient BMIs. For example, a first version can use foam with a first density of 4 lb/ft³ (such as foam 274) and a second version can use foam with a second density of 3 lb/ft³ (such as foam 266). Furthermore, the foam can be a polyurethane foam. The use of foam can be advantageous in that it can allow the contraction adjustment support 210 to be compressed and vacuum packed. This can reduce the required storage space and increase the accessibility of the contraction adjustment support 210, which can in turn encourage healthcare providers to utilize the device, increasing use and compliance.

In some examples, the contraction adjustment support 210 can be configured to elevate the body 110 between 1 inch to 2 inches from the surface on which the extremity offloading system 200 rests. For example, the maximum height or distance between the concave top surface and the convex bottom surface may be between 1 inch to 2 inches. The height at the center of the contraction adjustment support 210 can be between 1 inch to 2 inches. In some examples, the contraction adjustment support 210 can have a maximum depth between 9 to 10 inches. In some examples, the contraction adjustment support 210 can have a maximum width between 7 to 8 inches. It should be understood that different dimensions are possible for the contraction adjustment support 210, such as different heights or widths or different outer diameters.

### Foot-Ankle Positioner

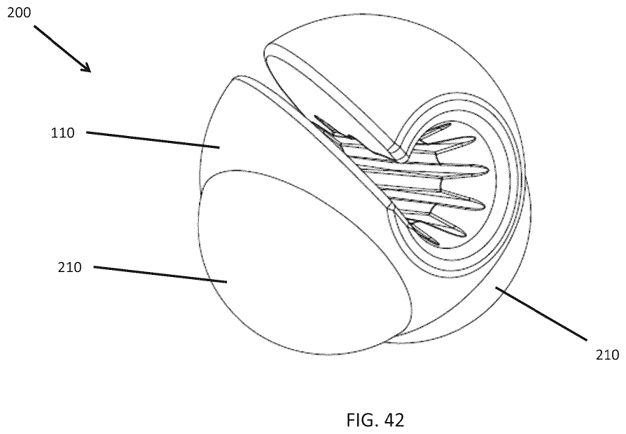

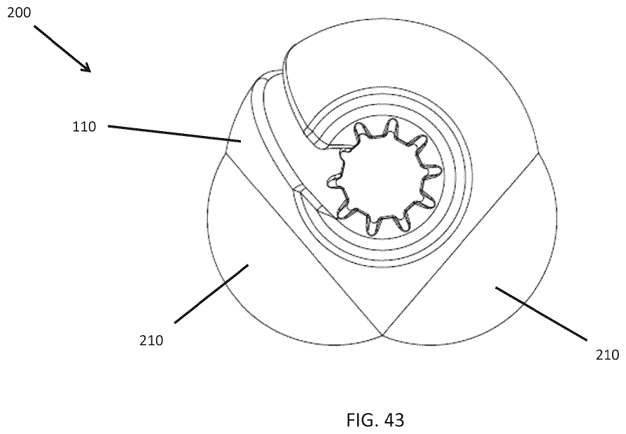

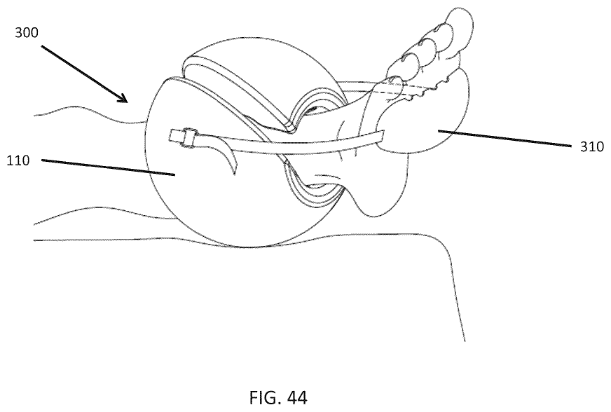

As shown in FIGS. 44-49, the extremity offloading system 300 can include a foot-ankle positioner 310, which can also be considered a foot support or foot plate 310. The foot-ankle positioner 310 can, at least partially, support a foot, an ankle, or both an ankle and a foot of a user. In FIGS. 44-49, like numbers are used to refer to parts similar to those of FIGS. 9-35 and reference can be made to the description of those parts made with reference to FIGS. 9-35. As with the embodiments described with reference to FIGS. 9-35 the extremity offloading system 300 can include a body 110, which can have a substantially spherical shape. In the illustrated embodiments, the extremity offloading system 300 may be configured similarly to the extremity offloading system 100 of FIGS. 9-35. Additionally, the extremity offloading system 300 of FIGS. 44-49 can include a foot support 310.

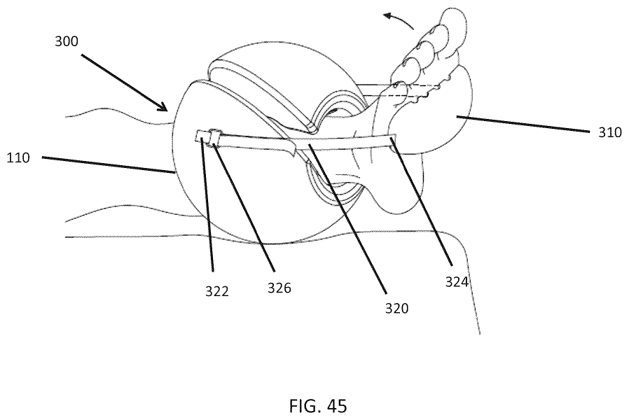

The foot support 310 can be configured to support and position the foot to prevent contracture of the Achilles tendon. The foot support 310 can be attached to the body 110. In some examples, the foot support 310 can be attached to the body 110 with at least one strap. As shown in FIGS. 44 and 45, the foot support 310 can be attached to the body 110 with two straps 320, one on each side of the foot support 310. Each strap 320 can have a first end 322 attached to the body 110 and a second end 324 attached to the foot support 310. The first end 322 of each of the two straps 320 can be positioned on either side of the opening 126 of the body 110. It should be understood that more than one or two straps 320 may be used. For example, the foot support 310 can be attached to the body 110 using 3, 4, or more straps. Further, attachment mechanisms other than straps may be used. For example, the foot support 310 can be attached to the body 110 using mesh, netting, cables, rods, or any other structure that may be used to affix the foot support 310 to the body 110.

The first end 322 of the strap 320 can be attached to the body 110 by an attachment mechanism. The attachment mechanism may include an adhesive or any other type of single use or multi-use attachment mechanism. For example, the attachment mechanism can be an adhesive tape, glue, hook and loop fasteners (e.g., VELCRO®), magnetic fasteners, snap fasteners, screws, and the like. In some cases, multiple types of attachment mechanisms may be used. The second end 324 of the strap 320 can be attached to the foot support 310 by a similar or different attachment mechanism as the first end 322. In some examples, the second end 324 of the strap 320 can be integrated into the foot support 310 making an attachment mechanism optional. For example, the foot support 310 can include one or more channels configured to receive the one or more straps 320.

In some examples, the foot support 310 can be attached to the body 110 by a single strap positioned through a channel extending through the width of the foot support 310. In this manner, the two ends of the single strap can extend from openings of the channel on either side of the foot support 310 and be attached to the body 110. Each end of the strap can be attached to opposing sides of the body 110.

The one or more straps 320 can include an adjustment mechanism 326. The adjustment mechanism 326 can allow the one or more straps 320 to increase or decrease in length. This can in turn adjust the position of the foot support 310 relative to the user's foot and/or the body 110.

As shown in FIGS. 44 and 45, the foot support 310 can be positioned on a bottom surface or a plantar surface of the user's foot. During use, the straps 320 of the foot support can be positioned on a medial and lateral side of the user's leg. The straps 320 can be tightened as the foot support 310 is positioned against a bottom surface of the user's foot, such that the foot support 310 can cause the foot to dorsiflex and prevent the foot from moving out of this flexed position, such as shown in FIG. 45. This can advantageously allow the user's foot to be kept up and prevent the Achilles tendon from contracting. The foot support 310 can easily be installed by one person as it does not require the foot to be in position before initially installing the foot support 310. Rather, the foot support 310 can be initially positioned against the user's foot as shown in FIG. 44. The straps 320 can then be adjusted to position the foot in the flexed position as shown in FIG. 45. The foot support 310 can also be used while still maintaining exposure of the heel.

The foot support 310 can be made of a foam. In some examples, the foot support 310 can be constructed from any material, such as foam. In some examples, the foot support 310 can be made of memory foam. In some examples, the foot support 310 can be made of memory foam. In some examples, the foot support 310 can be made of an antimicrobial or antibacterial material. In some examples, the material of the foot support 310 may include holes or may be made of a breathable material for the patient's comfort and/or to reduce or prevent an occurrence of infection. In some examples, the foot support 310 can be coated in a layer of an antimicrobial or antibacterial material. In some examples, the foot support 310 can be made of a material that is latex free, which advantageously makes the extremity offloading system 300 biocompatible. In some examples, the foot support 310 can be made of or coated with a water repellent material or coating. In some examples, the foot support 310 can be made of a foam with a density between 1 lb/ft³ to 26 lb/ft³, and in some examples between 1 lb/ft³ to 10 lb/ft³ or 3 lb/ft³ to 6 lb/ft³. In some examples, the foot support 310 can be made of the same or similar material as the body 110. In some examples, the foot support 310 can be made with a foam of a density between 3 lb/ft³ to 4 lb/ft³. Similar to the systems 10, 100 described above, the density of the foot support 310 can be based on the patient's BMI. Higher densities can be used for high patient BMIs. For example, a first version can use foam with a first density of 4 lb/ft³ (such as foam 274) and a second version can use foam with a second density of 3 lb/ft³ (such as foam 266). Furthermore, the foam can be a polyurethane foam. The use of foam can be advantageous in that it can allow the foot support 310 to be compressed and vacuum packed. This can reduce the required storage space and increase the accessibility of the foot support 310, which can in turn encourage healthcare providers to utilize the device, increasing use and compliance.

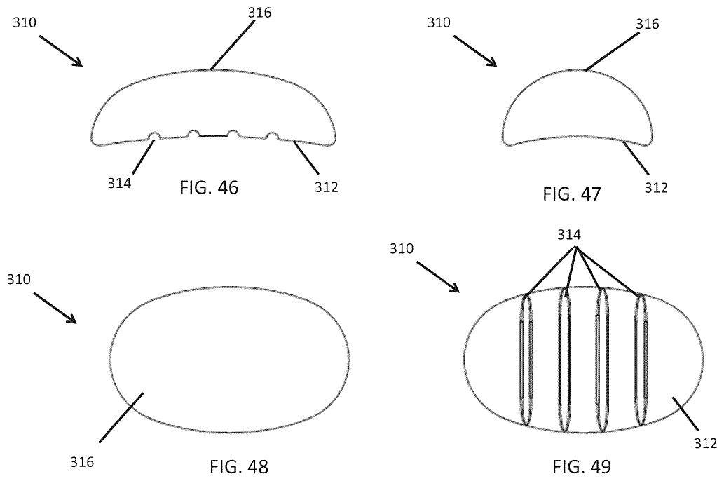

As shown in FIGS. 46 and 47, the foot support 310 can have a bottom surface 316 that is round, or at least partially round. The bottom surface 316 can be convex. The foot support 310 can have a top surface 312 that is also round or at least partially round. The top surface 312 can be concave. The curvature of the concave top surface 312 can be substantially similar or the same as the curvature of the convex bottom surface 316. The concave top surface 312 can be configured to prevent the user's feet from sliding off the ends of the foot support 310. In the top and bottom view, as shown in FIGS. 48 and 49, the foot support 310 can have a generally oval or elliptical shape.

The top surface 312 can also have a plurality of grooves 314. As shown in FIG. 49, the plurality of grooves or flutes 314 can be cutouts that extend along the entire height or length of the top surface 312. In some examples, the foot support can have 2, 3, 4, 5, 6, or more flutes. As shown, the foot support 310 can have 4 flutes, which can advantageously provide adequate air flow and support, while preventing the creation of too many pressure points for patient comfort. The plurality of flutes 314 may be positioned along the top surface 312 of the foot support 310, such that each of the plurality of flutes are configured to be positioned against a patient's foot when the patient's foot is positioned against the foot support 310. These flutes 314 can advantageously allow air to flow through the flutes 314 to provide comfort to the patient while the device is in use. For example, the flutes 314 can allow air to flow which can prevent excessive friction from occurring between the top surface 312 and the patient's skin. The flutes 314 can also lower the temperature of the patient's foot with the foot plate 310 positioned thereon. The flutes 314 can also reduce moisture buildup of the patient with the device positioned thereon.

In some examples, the foot support 310 can have a width, measured along the top surface 312 between 4 inches to 5 inches. The foot support 310 can have a depth, measured along the top surface 312 between 2.5 inches to 3.5 inches. The foot support 310 can have a maximum height measured from the top surface 312 to a bottom surface 316 between 1.5 inches to 2.5 inches. The straps 320 can have a width between a ½ inch to 1 inch, and in some examples ¾ inches. It should be understood that different dimensions are possible for the foot support 310, such as different heights, widths, depths, or thicknesses.

### Sensors

In some embodiments, the extremity offloading system 10, 100, 200, 300 can include one or more sensors to monitor various parameters. For example, the extremity offloading system 10, 100, 200, 300 can include one or more sensors to monitor movement of the system, such as an accelerometer, a gyroscope, or a magnetometer, which can detect various movement of the patients, such as if the patient has gotten up, if the patient is walking, or if the patient has fallen down. In another example, the extremity offloading system 10, 100, 200, 300 can include one or more sensors to measure orientation of the system, such as an accelerometer, a gyroscope, or a magnetometer, which can detect orientation of the system. The one or more sensors can thereby assist a user or a caretaker in ensuring the system is correctly and remains correctly positioned on the patient's leg or ankle. The one or more sensors scan also assist a user or caretaker in ensuring the system is consistently offloading the heel. In another example, the extremity offloading system can also include monitoring one or more of EKG waves (such as QRS waves), blood flow, blood pressure, pressure, temperature, glucose levels, position, or any other parameters. In another configuration, the extremity offloading system can include one or more sensors for monitoring humidity or moisture.

The extremity offloading system 10, 100, 200, 300 can include a circuit 150, which can house several circuit elements. The circuit can be a flexible circuit, which can advantageously curve, such as curving to match the curvature of the body 110. The circuit elements can include one or more of a microcontroller, a memory, a wireless communication element (such as Bluetooth sensor), or one or more sensors. In some examples, the one or more sensors may include a temperature sensor, an accelerometer, and a gyroscope. In other examples, the one or more sensors can include an accelerometer, a gyroscope, a magnetometer, a temperature sensor, a flow sensor, a pressure sensor, a friction sensor, a temperature sensor, an EKG sensor, a blood flow sensor, a blood pressure sensor, a glucose sensor, a position sensor, or an orientation sensor. The circuit 150 can be positioned within the extremity offloading system 10, 100, 200, 300. For example, as shown in FIG. 9, the extremity offloading system 100 can have a cutout or pocket in the body 110 configured to receive the circuit 150. The circuit 150 can be positioned in various places within the extremity offloading system 10, 100. The circuit 150 can be positioned on the sides of the extremity offloading system 10, 100, which advantageously prevents the weight of patient's leg being positioned directly on top of the circuit 150.

In some examples, one or more of the sensors can be configured to be positioned near or be in contact with the patient. For example, the one or more sensors can include a temperature probe which is configured to extend from the main circuit board, the temperature sensor probe can be configured to measure a temperature of the patient. Other types of sensors may also be configured as a probe, such as a pressure sensor probe or a humidity sensor probe. In some examples, the one or more sensor probes that are positioned near or in contact with a patient can be covered and protected with a waterproof, breathable fabric material, such as polytetrafluoroethylene (PTFE), like Teflon.

In some examples, the circuit 150 may be a single board or a single piece. In other examples, the circuit 150 may be modular, such that it is made of several pieces. A modular circuit may include at least one piece that is removable from the at least one other piece of the circuit. The at least one piece can include one or more sensors. In this manner, the at least one piece can be removable from the system, which allows the one or more sensors to be changed or replaced. This advantageously allows for repair or to customize the sensors used in the system.

The circuit 150 can be powered by a power component, such as with a battery. The battery can be rechargeable. The battery can be recharged with a cable, such as with a port through a surface of the extremity offloading system 10, 100. The battery can also be rechargeable through motion or wirelessly.

The circuit 150 can include a wireless communication element, such as a Bluetooth sensor. In some examples, the wireless communication element can include a radio or an antenna or antenna array, which may be configured to communicate on a medical device communications band. The wireless communication element can be configured to wirelessly transmit sensor data from the one or more sensors to a computing device, such as a phone, a tablet, or a computer. In some examples, the data can be received and displayed on the computing device. In other examples, the data can be received by the computing device, which can then perform calculations with the data. The computing device can also process the data to present the data in different visual ways, such as graphically or in a chart.

The use of monitoring various parameters can advantageously allow for remote monitoring methods, especially in consideration to telehealth. In some embodiments, an intermittent compression device can be coupled with the extremity offloading system 10, 100. The use of the intermittent compression device and the extremity offloading system 10, 100 would reduce or prevent the chances of both deep vein thrombosis (DVT) and HAPIs. The intermittent compression device could be positioned on a leg of the patient, such as on the calf of the patient, where the intermittent compression device is proximal to the extremity offloading system 10 in use.

### Additional Embodiments

In some embodiments, the extremity offloading system 10, 100, 200, 300 can include a stretchable wrap or band that is positioned around the outer surface of the extremity offloading system. This wrap or band can include elastic strap edging that would provide added security of the extremity offloading system 10, 100, 200, 300 to the patient's leg. The wrap or band could also provide a non-abrasive material against the surface on which the extremity offloading system 10, 100, 200, 300 is positioned on.

In some examples, the extremity offloading system 10, 100, 200, 300 can include a cloth that extends from the first end or the second end of the extremity offloading system. This cloth may be used to secure to the patient's leg or foot. For example, the cloth may be a sock, a layer of cotton padding, or a layer of foam (such as memory foam). The cloth can be configured to cover a foot and may be used to further secure the extremity offloading system to the patient.

In some examples, the extremity offloading system 10, 100, 200, 300 can include a cloth or stocking that is configured to cover an inner surface 50, 130. The cloth can be configured to be positioned between the inner surface 50, 130 and the skin of a patient. This cloth can be used to prevent skin irritation of a patient and prevent slippage of the system from the patient.

In some examples, the cloth or stocking attached to an inner surface or extending from the first end or the second end can include the one or more sensors or circuit 150 as previously described herein.

### Conclusion

Embodiments of systems, components and methods of assembly and manufacture are described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “proximal,” “distal,” “lateral,” “medial,” “anterior,” “posterior,” “top,” “bottom,” “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import. The use of any of the terms are not intended to limit the directionality or orientation of the device.

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

What is claimed is:

1. An extremity offloading system comprising:
a body comprising an outer surface, an inner surface, and a thickness extending between the outer surface and the inner surface, the outer surface being at least partially round, the inner surface defining an aperture, the aperture configured to accept insertion of a leg or an ankle of a patient; and
a contraction adjustment support comprising a convex bottom surface and a concave top surface, the concave top surface configured to receive at least a portion of the body.

2. The extremity offloading system of claim 1, wherein the contraction adjustment support is removably attached to the body.

3. The extremity offloading system of claim 1, wherein the concave top surface comprises an adhesive that is configured to attach the concave top surface to the outer surface of the body.

4. The extremity offloading system of claim 3, wherein the adhesive comprises tape, glue, hook and loop fasteners, magnetic fasteners, or snap fasteners.

5. The extremity offloading system of claim 1, wherein the contraction adjustment support is configured to be positioned between the body and a surface on which the contraction adjustment support rests.

6. The extremity offloading system of claim 5, wherein the convex bottom surface is positioned on the surface on which the contraction adjustment support rests.

7. The extremity offloading system of claim 6, wherein the contraction adjustment support is configured to elevate the body between 3 mm to 4 mm from the surface on which the contraction adjustment support rests.

8. The extremity offloading system of claim 1, wherein the contraction adjustment support comprises foam.

9. An extremity offloading system comprising:
a body comprising an outer surface, an inner surface, and a thickness extending between the outer surface and the inner surface, the outer surface being at least partially round, the inner surface defining an aperture, the aperture configured to accept insertion of a leg or an ankle of a patient; and
a foot-ankle positioner attached to the body by at least one strap, the foot-ankle positioner configured to at least partially support a foot of the patient, the ankle of the patient, or the foot and the ankle of the patient.

10. The extremity offloading system of claim 9, wherein the at least one strap comprises a first end attached to the body and a second end attached to the foot-ankle positioner.

11. The extremity offloading system of claim 10, wherein each of the first end is attached to the body and the second end is attached to the foot-ankle positioner with an adhesive.

12. The extremity offloading system of claim 11, wherein the adhesive comprises tape, glue, hook and loop fasteners, magnetic fasteners, or snap fasteners.

13. The extremity offloading system of claim 9, wherein the at least one strap comprises two straps, a first strap on a first side of an opening of the body and a second strap on a second side of the opening of the body.

14. The extremity offloading system of claim 9, wherein a first end of the at least one strap is attached to a first side of the body and a second end of the at least one strap is attached on a second side of the body.

15. The extremity offloading system of claim 14, wherein the foot-ankle positioner comprises a channel extending through a width of the foot-ankle positioner, the channel configured to receive the at least one strap.

16. The extremity offloading system of claim 9, wherein the at least one strap comprises an adjustment mechanism.

17. The extremity offloading system of claim 9, wherein the foot-ankle positioner comprises a convex bottom surface and a concave top surface.

18. The extremity offloading system of claim 9, wherein a top surface of the foot-ankle positioner comprises a plurality of grooves.

19. The extremity offloading system of claim 18, wherein a length of each of the plurality of grooves extend along a length of the top surface of the foot-ankle positioner.

20. The extremity offloading system of claim 9, wherein the foot-ankle positioner comprises foam.
